(19) United States
(12) Patent Application Publication
(10) Pub. No.: US 2021/0353621 A1
(43) Pub. Date: Nov. 18, 2021

(54) METHODS OF TREATING PROSTATE CANCER

(71) Applicant: Arvinas Operations, Inc., New Haven, CT (US)

(72) Inventors: Ronald PECK, Cheshire, CT (US); Yongqing HUANG, Flemington, NJ (US); Chi Fung KEUNG, Quincy, MA (US)

(21) Appl. No.: 17/317,235

(22) Filed: May 11, 2021

Related U.S. Application Data
(60) Provisional application No. 63/023,547, filed on May 12, 2020.

(51) Int. Cl. A61K 31/497; A61K 31/506; A61K 31/496; A61K 31/501; A61P 35/00

(52) U.S. Cl. CPC A61K 31/497; A61K 31/506; A61P 35/00; A61K 31/501; A61K 31/496

(57) ABSTRACT

The present application relates to treating and/or preventing prostate cancer, including metastatic and/or castrate-resistant prostate cancer, in a subject in need of treatment, comprising administering a compound of Formula (I),

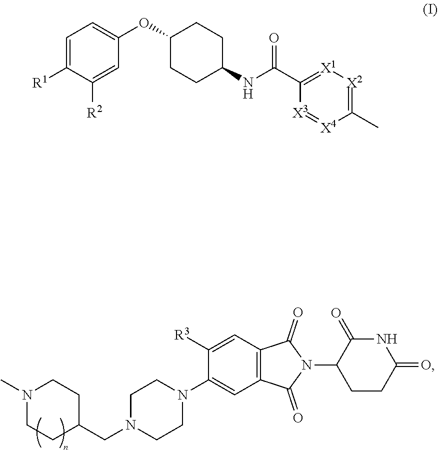

or a pharmaceutically acceptable salt, enantiomer, stereoisomer, solvate, polymorph, isotopic derivative, or prodrug thereof, wherein R¹, R², R³, X¹, X², X³, X⁴, and n are defined herein.

### RELATED APPLICATIONS

This application claims priority to, and the benefit of, U.S. Provisional Application No. 63/023,547, filed May 12, 2020, which is incorporated herein by reference in its entirety for all purposes.

### FIELD OF THE DISCLOSURE

This application relates to treating prostate cancer, including metastatic and/or castrate-resistant prostate cancer, comprising administering a compound of Formula (I) to a subject in need of treatment.

### BACKGROUND OF THE DISCLOSURE

Androgen Receptor (AR) belongs to a nuclear hormone receptor family that is activated by androgens, such as testosterone and dihydrotestosterone (Pharmacol. Rev. 2006, 58(4), 782-97; Vitam. Horn. 1999, 55:309-52). In the absence of androgens, AR is bound by Heat Shock Protein 90 (Hsp90) in the cytosol. When an androgen binds AR, its conformation changes to release AR from Hsp90 and to expose the Nuclear Localization Signal (NLS). The latter enables AR to translocate into the nucleus where AR acts as a transcription factor to promote gene expression responsible for male sexual characteristics (Endocr. Rev. 1987, 8(1): 1-28; Mol. Endocrinol. 2002, 16(10), 2181-7). AR deficiency leads to Androgen Insensitivity Syndrome, formerly termed testicular feminization.

While AR is responsible for development of male sexual characteristics, it is also a well-documented oncogene in certain forms of cancers including prostate cancers (Endocr. Rev. 2004, 25(2), 276-308). A commonly measured target gene of AR activity is the secreted Prostate Specific Antigen (PSA) protein. The current treatment regimen for prostate cancer involves inhibiting the androgen-AR axis by two methods. The first approach relies on reduction of androgens, while the second strategy aims to inhibit AR function (Nat. Rev. Drug Discovery, 2013, 12, 823-824). Despite the development of effective targeted therapies, most patients develop resistance and the disease progresses. An alternative approach for the treatment of prostate cancer involves eliminating the AR protein. Because AR is a critical driver of tumorigenesis in many forms of prostate cancers, its elimination should lead to a therapeutically beneficial response. There exists an ongoing need in the art for effective treatments for diseases, especially cancer, prostate cancer, and Kennedy's Disease. In addition, the advances here improve patient safety.

### SUMMARY OF THE DISCLOSURE

In one aspect, this application pertains to a method of treating prostate cancer in a subject in need thereof, comprising administering to the subject a therapeutically effective amount of a compound of Formula (I),

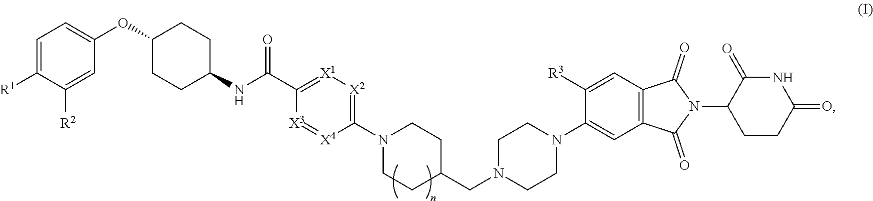

or a pharmaceutically acceptable salt, enantiomer, stereoisomer, solvate, polymorph, isotopic derivative, or prodrug thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH; and
   further comprising the step of discontinuing or reducing the administration of rosuvastatin to the subject prior to initiating administration of the compound of Formula (I).

In one aspect, this application pertains to a method of treating prostate cancer in a subject in need thereof, comprising administering to the subject a therapeutically effective amount of a compound of Formula (I),

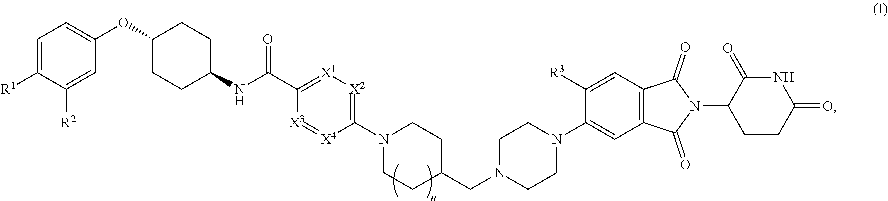

wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH; and
   further comprising the step of discontinuing or reducing the administration of rosuvastatin to the subject prior to initiating administration of the compound of Formula (I).

In one embodiment, the prostate cancer is castrate-resistant prostate cancer.

In one embodiment, the prostate cancer is metastatic prostate cancer.

In one embodiment, R¹ is CN and R² is chloro.

In one embodiment, R³ is hydrogen.

In one embodiment, R³ is fluoro.

In one embodiment, n is 0.

In one embodiment, n is 1.

In one embodiment, each of X¹, X², X³, and X⁴ is CH.

In one embodiment, three of X¹, X², X³, and X⁴ are CH and the other is N.

In one embodiment, two of X¹, X², X³, and X⁴ are CH and the other two are N.

In one embodiment, the compound of Formula (I) is selected from the group consisting of:

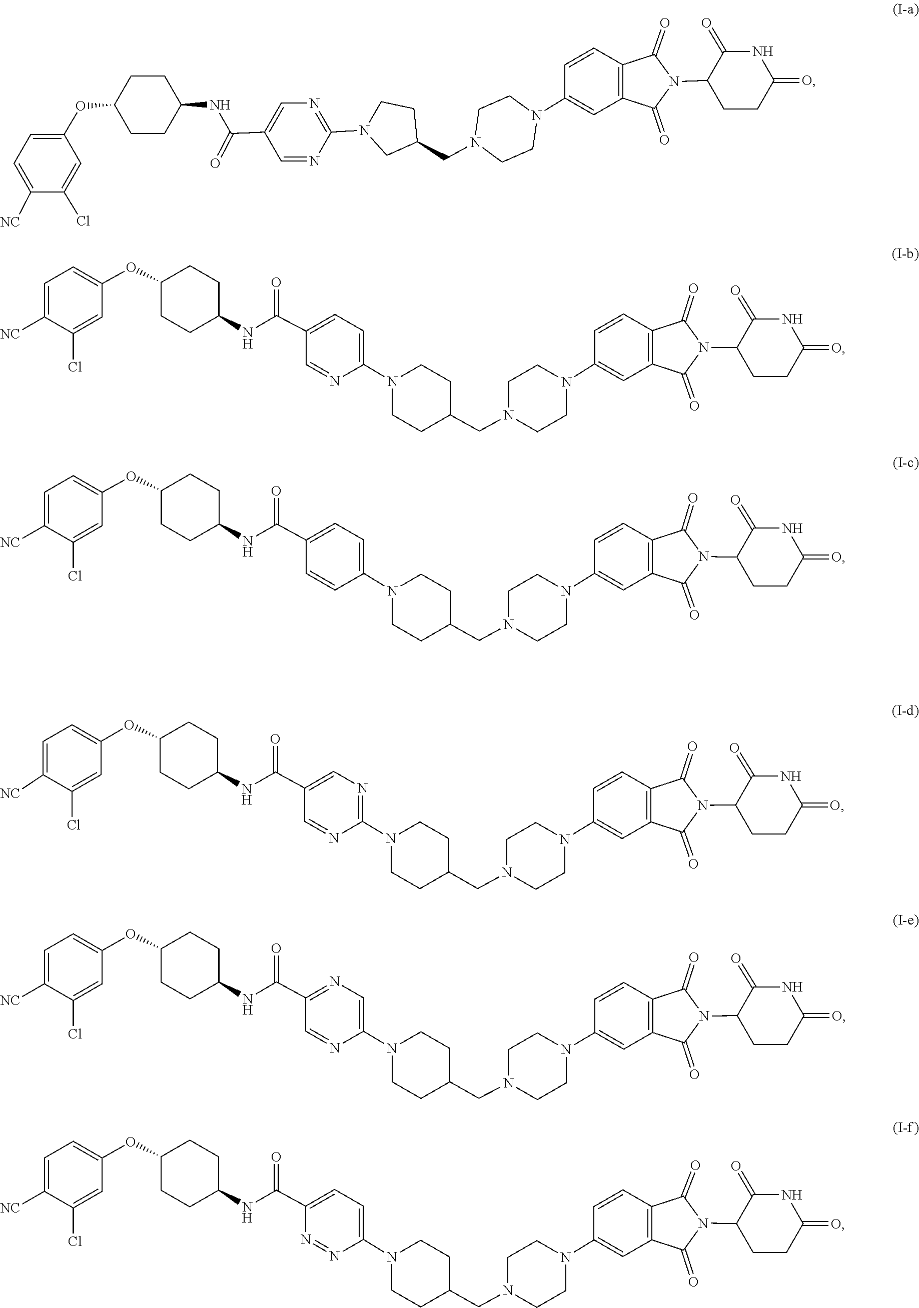

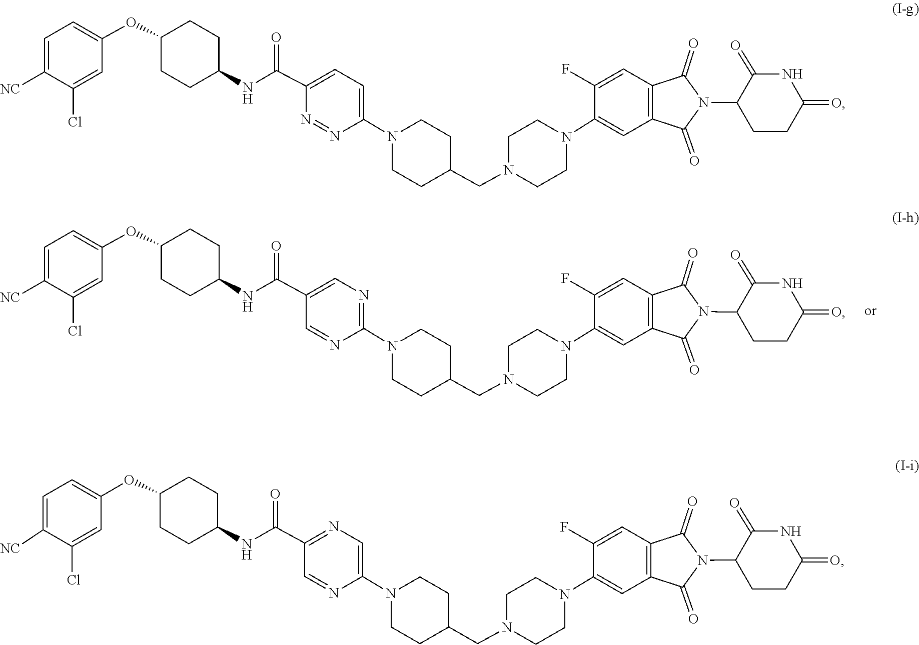 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "or" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

or a pharmaceutically acceptable salt, enantiomer, stereoisomer, solvate, polymorph, isotopic derivative, or prodrug thereof.

In one embodiment, the compound of Formula (I) is selected from the group consisting of:

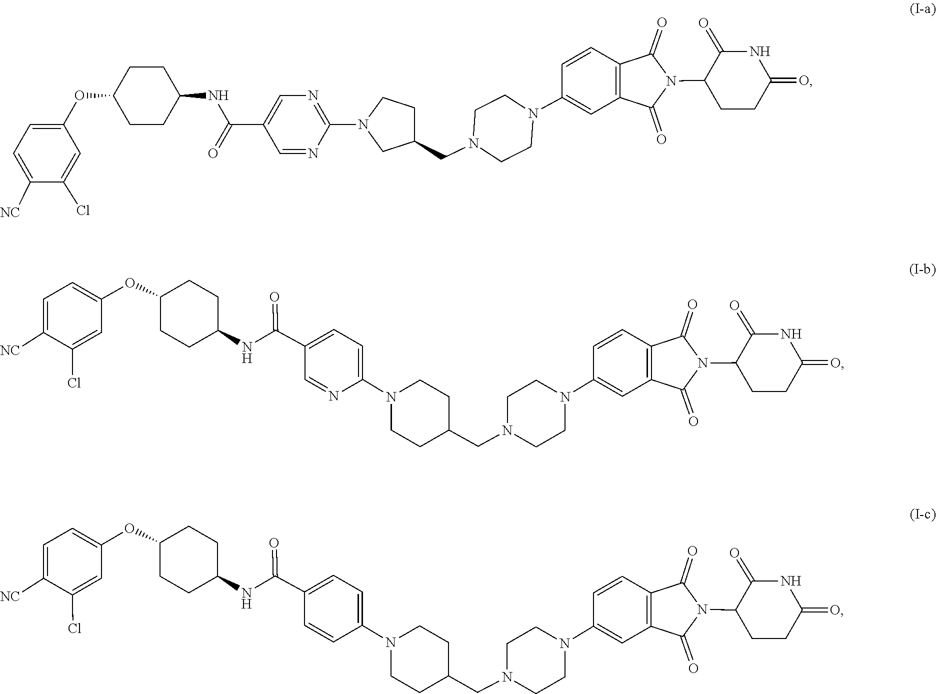 [SMILES: N#Cc1ccc(O[C@H]2CC[C@H](NC(=O)c3cnc(N4CC[C@@H](CN5CCN(c6ccc7c(c6)C(=O)N(C6CCC(=O)NC6=O)C7=O)CC5)C4)nc3)CC2)cc1Cl ; N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)nc3)CC2)cc1Cl ; N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)cc3)CC2)cc1Cl]

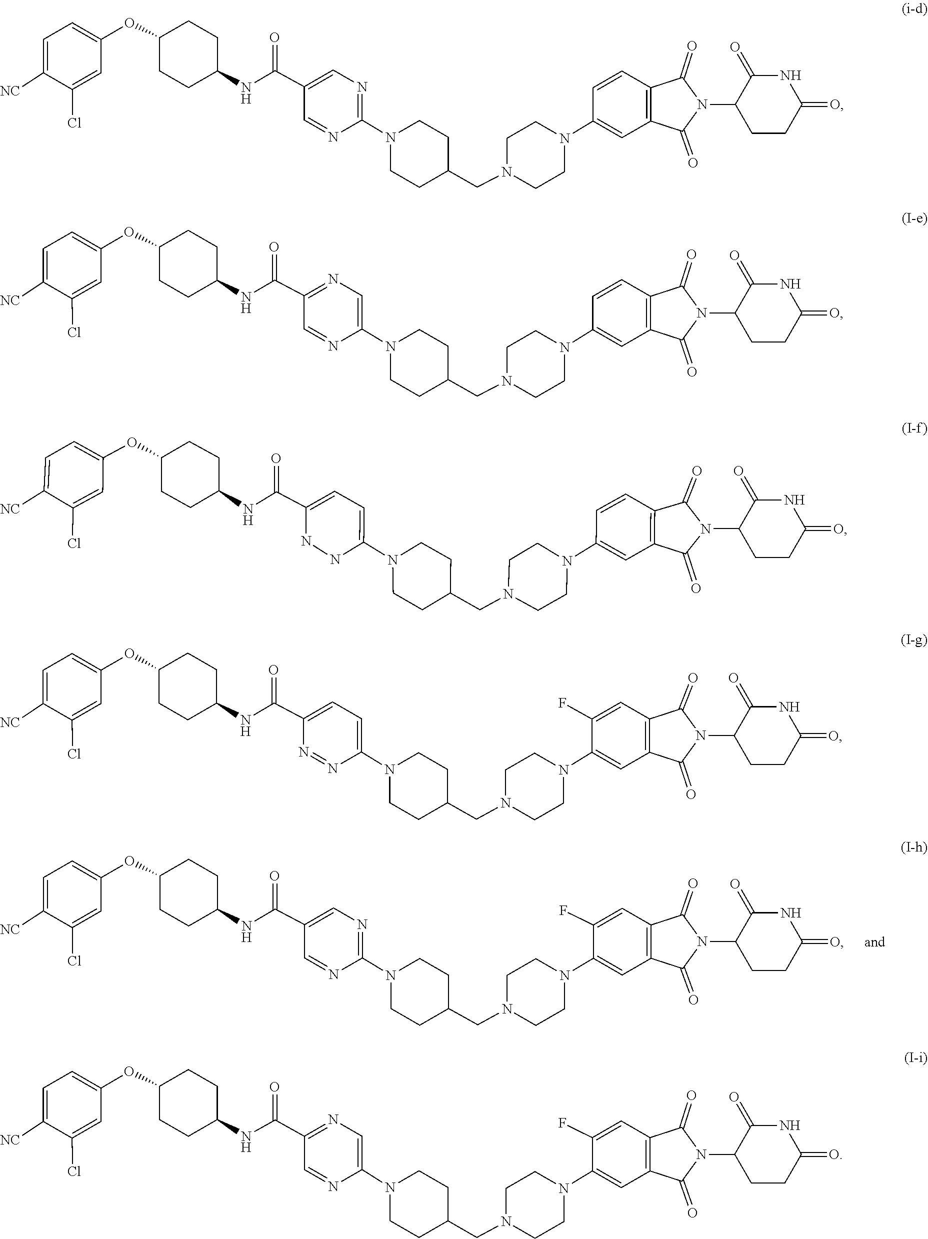

In one embodiment, the compound of Formula (I) is administered orally to the subject.

In one embodiment, the compound of Formula (I) is administered orally to the subject, but the subject should not initiate, continue, or maintain the same level of treatment with the BCRP substrate rosuvastatin while taking any therapy that includes Compound (I-g).

In one embodiment, the therapeutically effective amount of the compound of Formula (I) is administered to the subject once a day, twice a day, three times a day, or four times a day. In one embodiment, the therapeutically effective amount of the compound of Formula (I) is administered to the subject once a day. In one embodiment, the therapeutically effective amount of the compound of Formula (I) is administered to the subject all at once or is administered in two, three, or four portions.

In one embodiment, the therapeutically effective amount of the compound of Formula (I) is about 70 mg to about 1000 mg.

In one embodiment, the therapeutically effective amount of the compound of Formula (I) is about 100 mg to about 280 mg.

In one embodiment, the therapeutically effective amount of the compound of Formula (I) results in a mean day 15 AUC₀₋₂₄ of greater than about 4,500 ng*hr/mL, or greater than about 4,600 ng*hr/mL, about 4,700 ng*hr/mL, about 4,800 ng*hr/mL, about 4,900 ng*hr/mL, about 5,000 ng*hr/mL, about 5,100 ng*hr/mL, about 5,200 ng*hr/mL, about 5,300 ng*hr/mL, 5,400 ng*hr/mL, about 5,500 ng*hr/mL, about 5,600 ng*hr/mL, about 5,700 ng*hr/mL, about 5,800 ng*hr/mL, about 5,900 ng*hr/mL, or about 6,000 ng*hr/mL.

In one embodiment, the therapeutically effective amount of the compound of Formula (I) results in a mean day 15 AUC₀₋₂₄ of greater than about 4,500 ng*hr/mL and less than about 5,500 ng*hr/mL.

In one embodiment, the therapeutically effective amount of the compound of Formula (I) results in a mean day 15 Cₘₐₓ of greater than about 300 ng/mL and less than about 400 ng/mL.

In one embodiment, the therapeutically effective amount of the compound of Formula (I) results in a mean day 15 Cₘₐₓ of greater than (as to all of the following) about 330 ng/mL, about 335 ng/mL, about 340 ng/mL, about 345 ng/mL, about 350 ng/mL, about 355 ng/mL, about 360 ng/mL, about 365 ng/mL, about 370 ng/mL, about 375 ng/mL, or about 380 ng/mL.

In one embodiment, the compound of Formula (I) is formulated as a tablet, capsule or oral liquid. In one embodiment, the compound of Formula (I) is formulated as a tablet. In one embodiment, the tablet comprises a compound of Formula (I) and, optionally, one or more of the following: emulsifier; surfactant; binder; disintegrant; glidant; and lubricant.

In one embodiment, the subject in need of treatment is in a fed state at the time of administration of the compound.

In one embodiment, the subject in need of treatment is in a fasted state at the time of administration of the compound.

In one aspect, this application pertains to a method of treating prostate cancer in a subject in need thereof, comprising once a day, oral administration of a therapeutically effective amount of the compound of Formula (I), or a pharmaceutically acceptable salt, enantiomer, stereoisomer, solvate, polymorph, isotopic derivative, or prodrug thereof, wherein the compound of Formula (I) is selected from the group consisting of:

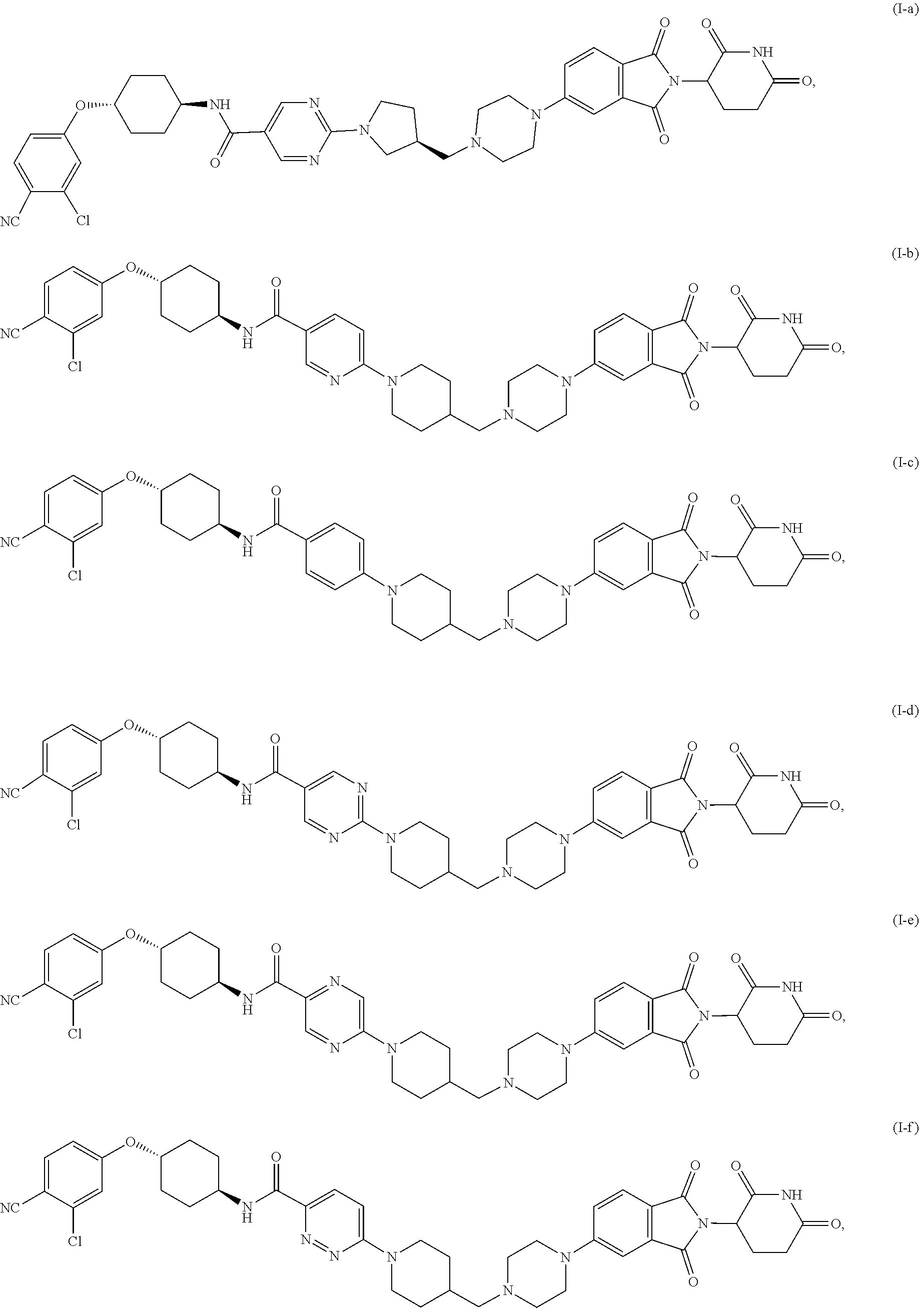

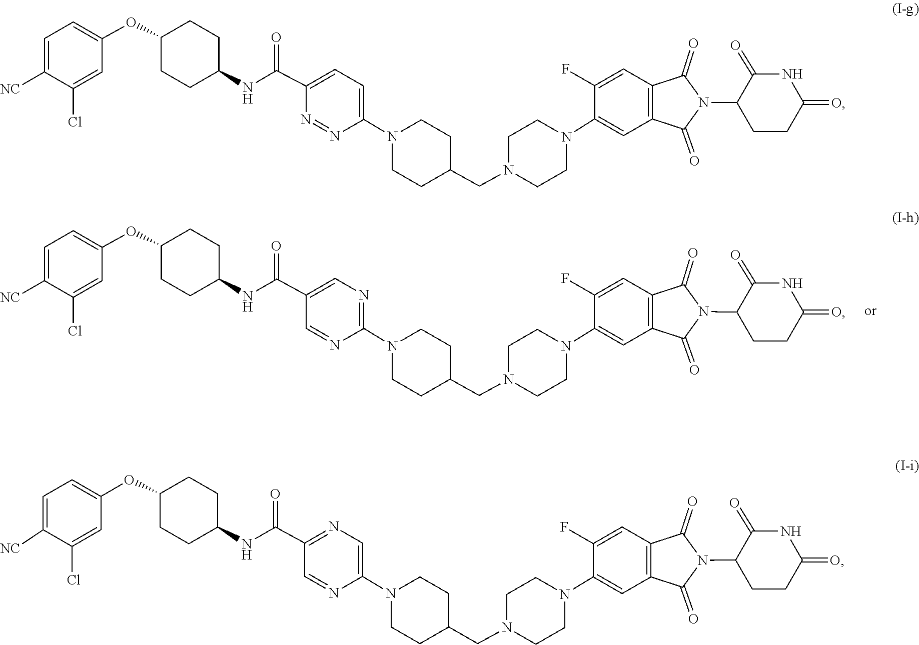 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "or" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

or a pharmaceutically acceptable salt, enantiomer, stereoisomer, solvate, polymorph, isotopic derivative, or prodrug thereof,
further comprising the step of discontinuing or reducing the administration of rosuvastatin to the subject prior to initiating administration of the compound of Formula (I).

In one aspect, this application pertains to a method of treating prostate cancer in a subject in need thereof, comprising once a day, oral administration of a therapeutically effective amount of the compound of Formula (I), wherein the compound of Formula (I) is selected from the group consisting of:

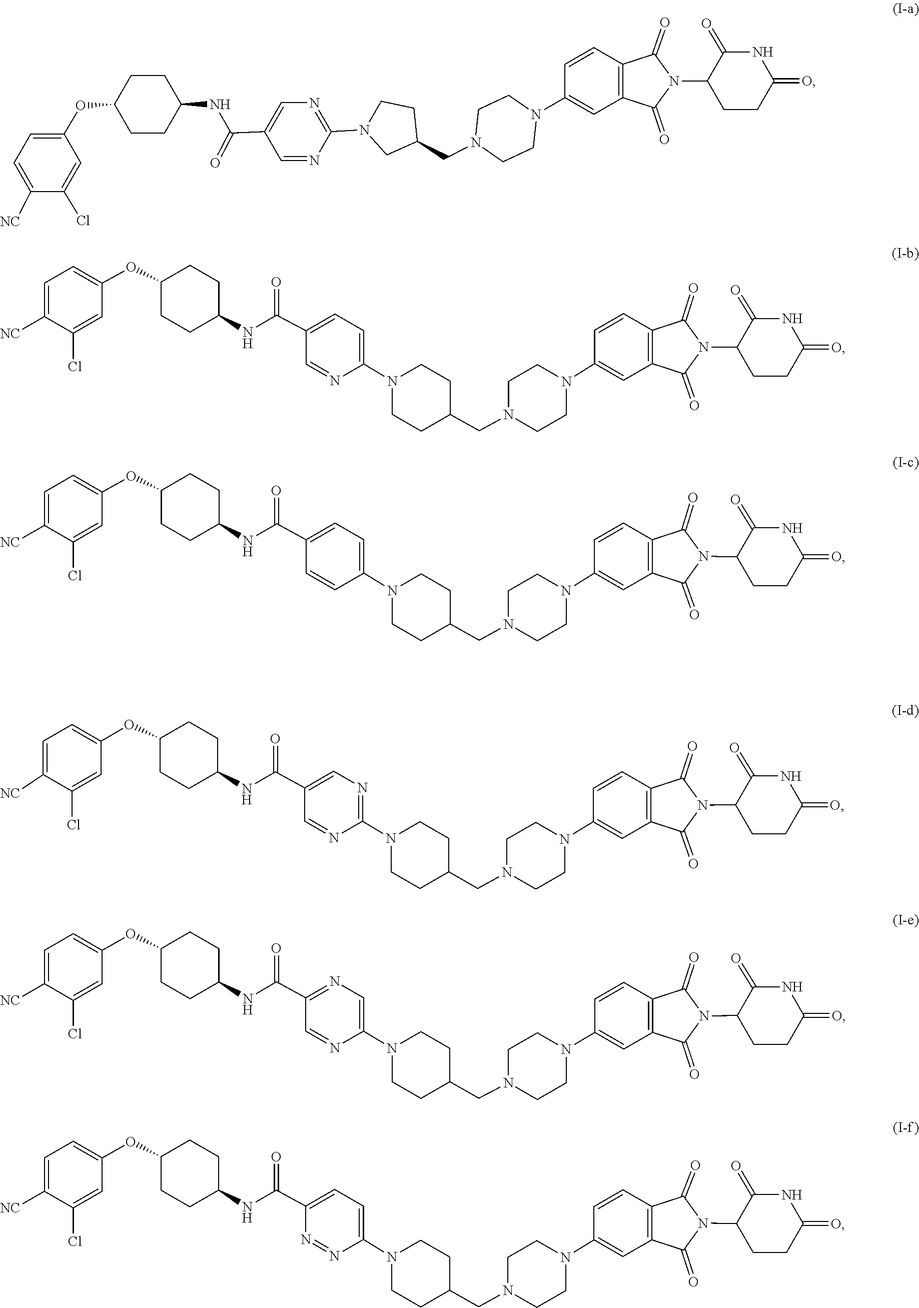

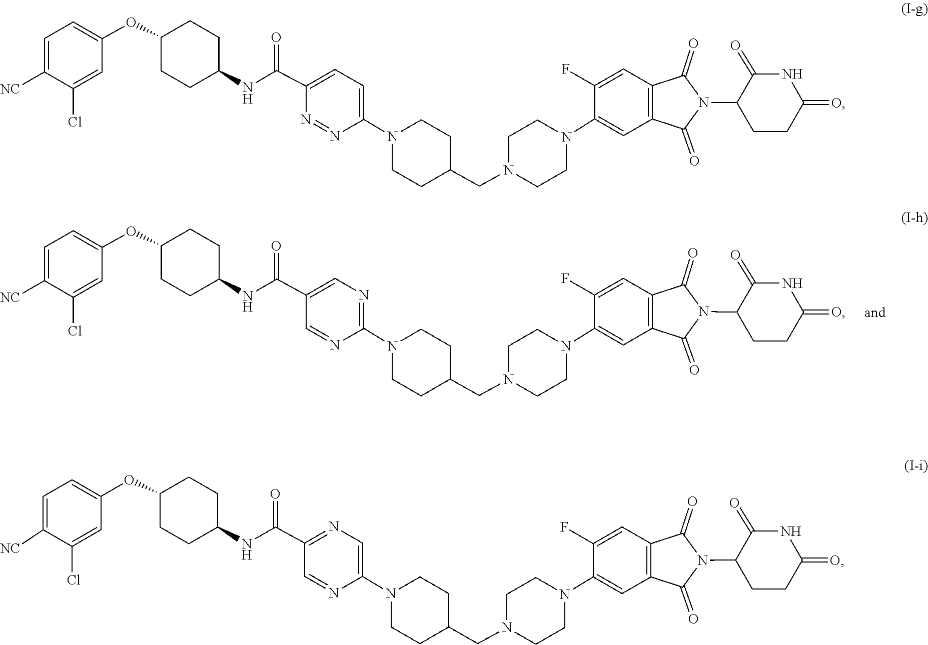 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "and" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

further comprising the step of discontinuing or reducing the administration of rosuvastatin to the subject prior to initiating administration of the compound of Formula (I).

In one embodiment, the therapeutically effective amount of the compound of Formula (I) is administered to the subject all at once or is administered in two, three, or four portions.

In one embodiment, the therapeutically effective amount of the compound of Formula (I) is about 70 mg to about 1000 mg.

In one embodiment, the compound of Formula (I) is formulated as a tablet.

In one embodiment, the method comprises the step of discontinuing the administration of rosuvastatin to the subject prior to initiating administration of a therapeutically effective amount of the compound of Formula (I), or a pharmaceutically acceptable salt, enantiomer, stereoisomer, solvate, polymorph, isotopic derivative, or prodrug thereof.

In one embodiment, the administration of rosuvastatin is discontinued at a time point that is at least the longer of 5 days or 5 half-lives of rosuvastatin prior to initiating the administration of compound (I), or a pharmaceutically acceptable salt, enantiomer, stereoisomer, solvate, polymorph, isotopic derivative, or prodrug thereof.

In one embodiment, the method comprises the step of reducing the administration of rosuvastatin to the subject prior to initiating administration of a therapeutically effective amount of the compound of Formula (I), or a pharmaceutically acceptable salt, enantiomer, stereoisomer, solvate, polymorph, isotopic derivative, or prodrug thereof.

In one embodiment, the administration of rosuvastatin is reduced at a time point that is at least the longer of 5 days or 5 half-lives of rosuvastatin prior to initiating the administration of compound (I), or a pharmaceutically acceptable salt, enantiomer, stereoisomer, solvate, polymorph, isotopic derivative, or prodrug thereof.

In one embodiment, the compound of Formula (I) is the compound (I-g).

In one aspect, the application pertains to a use of a compound of Formula (I) for the manufacture of a medicament for treatment of prostate cancer, wherein the medicament includes a therapeutically effective amount of a compound of Formula (I),

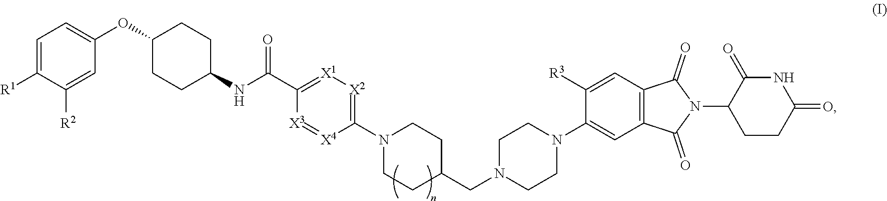

or a pharmaceutically acceptable salt, enantiomer, stereoisomer, solvate, polymorph, isotopic derivative, or prodrug thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH;
wherein the medicament is administered to a subject who is also discontinuing or reducing the use of prior to initiating administration of the medicament.

In one aspect, the application pertains to a use of a compound of Formula (I) for the manufacture of a medicament for treatment of prostate cancer, wherein the medicament includes a therapeutically effective amount of a compound of Formula (I),

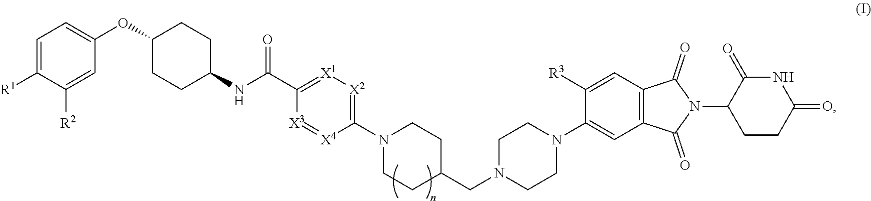

wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH;
wherein the medicament is administered to a subject who is also discontinuing or reducing the use of prior to initiating administration of the medicament.

In one embodiment, the medicament is administered to a subject who is also discontinuing the use of rosuvastatin prior to initiating administration of the medicament.

In one embodiment, the medicament is administered to a subject who is also reducing the use of rosuvastatin prior to initiating administration of the medicament.

In one embodiment, the use of rosuvastatin is discontinued beginning at a time point prior to initiating the administration of the medicament, wherein the time point is at least the longer of 5 days or 5 half-lives of rosuvastatin.

In one embodiment, the use of rosuvastatin is reduced beginning at a time point prior to initiating the administration of the medicament, wherein the time point is at least the longer of 5 days or 5 half-lives of rosuvastatin.

In one embodiment, the medicament comprises a compound of Formula (I) that is the compound (I-g).

### BRIEF DESCRIPTION OF THE FIGURES

FIG. 1 is a dose-response curve comparing the in vitro inhibitory effect of VCaP proliferation of Compound (I-g) with enzalutamide.

FIG. 2 is Western Blot experiment that shows the reduction of AR in VCaP tumor cells in response to treatment with Compound (I-g) at concentrations of 0.03 nM, 0.1 nM, 0.3 nM, 1 nM, 3 nM, 10 nM, 30 nM, 100 nM, and 300 nM.

FIG. 3 is series of line graphs summarizing animal experiments performed in a castrated VCaP xenograft model. Compound (I-g) was administered orally, once daily at doses of 0.1 mg/kg (mpk), 0.3 mg/kg, 1 mg/kg, and 3 mg/kg. Enzalutamide (20 mg/kg) and vehicle were also used as control groups.

FIG. 4 is series of line graphs summarizing animal experiments performed in an intact (non-castrated) VCaP xenograft model. Compound (I-g) was administered orally, once daily at doses of 1 mg/kg, 3 mg/kg, and 10 mg/kg. Enzalutamide (20 mg/kg) and vehicle were also used as control groups.

FIG. 5 is series of line graphs summarizing animal experiments performed in an enzalutamide resistant VCaP xenograft model. Compound (I-g) was administered orally, once daily at doses of 3 mg/kg and 10 mg/kg. Enzalutamide (20 mg/kg) and vehicle were also used as control groups.

FIG. 6 is Western Blot experiment that shows the reduction of AR in enzalutamide-resistant VCaP tumors in response to dosing with Compound (I-g) at 10 mg/kg and 3 mg/kg (oral, once daily).

FIG. 7 is a series of line graphs which provides a representation of the mean concentrations of Compound (I-g) over a 24 hour time period after dosing on day 15 for all three tested doses (35 mg/day, 70 mg/day, and 140 mg/day, oral administration).

### DETAILED DESCRIPTION

### Definitions

“Halogen” or “halo” refers to fluorine (F), chlorine (Cl), bromine (Br), or iodine (I).

“C₁-C₆ alkyl” refers to a straight or branched chain saturated hydrocarbon containing 1-6 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Pharmaceutically acceptable salt”, as used herein with respect to a compound of Formula (I), means a salt form of a compound of Formula (I) as well as hydrates of the salt form with one or more water molecules present. Such salt and hydrated forms retain the biological activity of a compound of Formula (I) and are not biologically or otherwise undesirable, i.e., exhibit minimal, if any, toxicological effects. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methyl sulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “isomer” refers to salts and/or compounds that have the same composition and molecular weight but differ in physical and/or chemical properties. The structural difference may be in constitution (geometric isomers) or in the ability to rotate the plane of polarized light (stereoisomers). With regard to stereoisomers, the salts of a compound of Formula (I) may have one or more asymmetric carbon atom and may occur as racemates, racemic mixtures and as individual enantiomers or diastereomers.

The compounds of Formula (I) may exist in unsolvated as well as solvated forms such as, for example, hydrates.

“Solvate” means a solvent addition form that contains either a stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate.

Hydrates are formed by the combination of one or more molecules of water with one of the substances in which the water retains its molecular state as H₂O, such combination being able to form one or more hydrate. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Equally suitable are the hydrates of salts of the compounds of the invention.

When a compound is crystallized from a solution or slurry, it can be crystallized in a different arrangement lattice of spaces (this property is called “polymorphism”) to form crystals with different crystalline forms, each of which is known as “polymorphs”. “Polymorph”, as used herein, refers to a crystal form of a compound of Formula (I) where the molecules are localized in the three-dimensional lattice sites. Different polymorphs of the compound of Formula (I) may be different from each other in one or more physical properties, such as solubility and dissolution rate, true specific gravity, crystal form, accumulation mode, flowability and/or solid state stability, etc.

“Isotopic derivative”, as referred to herein, relates to a compound of Formula (I) that is isotopically enriched or labelled (with respect to one or more atoms of the compound) with one or more stable isotopes. Thus, in this application, the compounds of Formula (I) include, for example, compounds that are isotopically enriched or labelled with one or more atoms such as deuterium.

The term “pharmaceutically acceptable prodrugs” as used herein refers to those prodrugs of the compounds of Formula (I) which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention.

“Prodrug”, as used herein means a compound which is convertible in vivo by metabolic means (e.g., by hydrolysis) to afford any compound delineated by the formulae of the instant invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 1 13-191 (1991); Bundgaard, et al., Journal of Drug Deliver Reviews, 8:1-38 (1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

This invention also encompasses pharmaceutical compositions containing, and methods of treating disorders through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1 15. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities. Combinations of substituents and variables envisioned by this invention are only those that result in the formation of stable compounds.

Metastatic prostate cancer, or metastases, refers to prostate cancer that has spread beyond the prostate to other parts of the body, e.g., bones, lymph nodes, liver, lungs, brain.

Castrate-resistant prostate cancer or castration-resistant prostate cancer (or prostate cancer that is castrate- or castration-resistant) is a type of prostate cancer that keeps growing even when the amount of testosterone in the body is reduced to very low levels.

Metastatic, castrate-resistant prostate cancer is a type of prostate cancer that has metastasized and continues to grow even when the amount of testosterone in the body is reduced to very low levels.

As used herein, “treating” describes the management and care of a subject for the purpose of combating a disease, condition, or disorder and includes decreasing or alleviating the symptoms or complications, or eliminating the disease, condition or disorder.

As used herein, “preventing” describes stopping the onset of the symptoms or complications of the disease, condition or disorder.

“Administration” refers to introducing an agent, such as a compound of Formula (I) into a subject. The related terms “administering” and “administration of” (and grammatical equivalents) refer both to direct administration, which may be administration to a subject by a medical professional or by self-administration by the subject, and/or to indirect administration, which may be the act of prescribing a drug. For example, a physician who instructs a patient to self-administer a drug and/or provides a patient with a prescription for a drug is administering the drug to the patient.

“Therapeutically effective amount”, as used herein means an amount of the free base of a compound of Formula (I) that is sufficient to treat, ameliorate, or prevent a specified disease (e.g., prostate cancer), disease symptom, disorder or condition, or to exhibit a detectable therapeutic or inhibitory effect. The effect can be detected by any assay method known in the art. The effective amount for a particular subject may depend upon the subject's body age, gender, weight, size, and health; the nature and extent of the condition; and whether additional therapeutics are to be administered to the subject. Therapeutically effective amounts for a given situation can be determined by routine experimentation that is within the skill and judgment of the clinician.

“Cₘₐₓ”, as used herein, refers to the observed maximum (peak) plasma concentration of a specified compound in the subject after administration of a dose of that compound to the subject.

“AUC”, as used herein, refers to the total area under the plasma concentration-time curve, which is a measure of exposure to a compound of interest, and is the integral of the concentration-time curve after a single dose or at steady state. AUC is expressed in units of ng*H/mL (ng×H/mL).

“AUCₜₐᵤ”, as used herein, refers to the AUC from 0 hours to the end of a dosing interval.

“AUC₀₋₂₄” means the AUC from 0 hours to 24 hours after administration of a single dose.

“Controlled release” or “CR” as used herein with respect to an oral dosage form of the disclosure means that a compound of Formula (I) is released from the dosage form according to a pre-determined profile that may include when and where release occurs after oral administration and/or a specified rate of release over a specified time period.

“Controlled release agent” as used herein with respect to an oral dosage form of the disclosure refers to one or more substances or materials that modulate release of a compound of Formula (I) from the dosage form. Controlled release agents may be materials which are organic or inorganic, naturally occurring or synthetic, such as polymeric materials, triglycerides, derivatives of triglycerides, fatty acids and salts of fatty acids, talc, boric acid and colloidal silica.

“Enteric coating” as used herein with respect to a dosage form of the disclosure refers to a pH-dependent material that surrounds a core comprising a compound of Formula (I) and which remains substantially intact in the acid environment of the stomach, but which dissolves in the pH environment of the intestines.

“Gastro-resistant” or “GR” as applied to a CR oral dosage form described herein means that release of a compound of Formula (I) in the stomach of a subject shall not exceed 5%, 2.5%, 1% or 0.5% of the total amount of the compound of Formula (I) in the dosage form.

“Oral dosage form” as used herein refers to a pharmaceutical drug product that contains a specified amount (dose) of a compound of Formula (I) as the active ingredient, or a pharmaceutically acceptable salt and/or solvate thereof, and inactive components (excipients), formulated into a particular configuration that is suitable for oral administration, such as a tablet, capsule or oral liquid. In one embodiment, the compositions are in the form of a tablet that can be scored.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

Abiraterone acetate is a commercially available drug for the treatment of metastatic castration-resistant prostate cancer developed by Janssen and sold under the brand name Zytiga®.

The term “about” as part of a quantitative expression such as “about X”, includes any value that is 10% higher or lower than X, and also includes any numerical value that falls between X−10% and X+10%. Thus, for example, a weight of about 40 g includes a weight of between 36 to 44 g.

“Comprising” or “comprises” as applied to a particular dosage form, composition, use, method or process described or claimed herein means that the dosage form, composition, use, method, or process includes all of the recited elements in a specific description or claim, but does not exclude other elements. “Consists essentially of” and “consisting essentially of” means that the described or claimed composition, dosage form, method, use, or process does not exclude other materials or steps that do not materially affect the recited physical, pharmacological, pharmacokinetic properties or therapeutic effects of the composition, dosage form, method, use, or process. “Consists of” and “consisting of” means the exclusion of more than trace elements of other ingredients and substantial method or process steps.

“Fasted condition” or “fasted state” as used to describe a subject means the subject has not eaten for at least 4 hours before a time point of interest, such as the time of administering a compound of Formula (I). In an embodiment, a subject in the fasted state has not eaten for at least any of 6, 8, 10 or 12 hours prior to administration of a compound of Formula (I).

“Fed condition” or “fed state” as used to describe a subject herein means the subject has eaten less than 4 hours before a time point of interest, such as the time of administering a compound of Formula (I). In an embodiment, a subject in the fed state has not eaten for at least any of 3, 2, 1 or 0.5 hours prior to administration of a compound of Formula (I).

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The terms “patient” and “subject” are used interchangeably herein, and refer to a mammal, e.g., a human, mouse, rat, guinea pig, dog, cat, horse, cow, pig, or non-human primate, such as a monkey, chimpanzee, baboon or rhesus.

In one embodiment, the subject is a human.

In one embodiment, the subject is a human who has been diagnosed with prostate cancer.

In one embodiment, the subject is a human who has been diagnosed with metastatic prostate cancer.

In one embodiment, the subject is a human who has been diagnosed with castrate-resistant prostate cancer.

In one embodiment, the subject is a human who has been diagnosed with metastatic, castrate-resistant prostate cancer.

### Compounds of Formula (I)

In one aspect, the application pertains to the methods of treating and/or preventing cancer comprising the administration of a compound of Formula (I) to a subject in need thereof. In one aspect, the application pertains to the use of a compound of Formula (I) in the treatment and/or prevention of prostate cancer. In one aspect, the application pertains to the use of a compound of Formula (I) in the manufacture of a medicament for the treatment and/or prevention of prostate cancer.

As referred to herein, a compound of Formula (I) refers to a compound with the following structure:

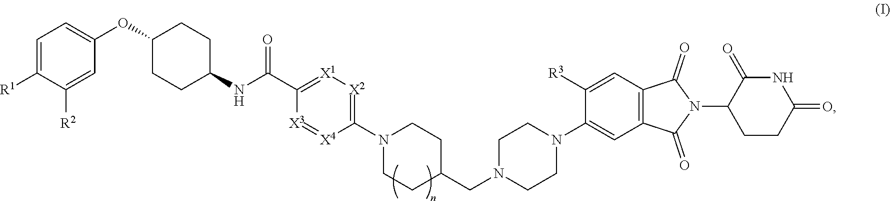

or a pharmaceutically acceptable salt, enantiomer, stereoisomer, solvate, polymorph, isotopic derivative, or prodrug thereof, wherein:

R¹ is hydrogen, CN, or C₁-C₆ alkyl;

R² is hydrogen, halo, or C₁-C₆ alkyl;

R³ is hydrogen or halo;

X¹ is CH or N;

X² is CH or N;

X³ is CH or N;

X⁴ is CH or N; and

n is 0 or 1;

provided that at least two of X¹, X², X³, and X⁴ are CH.

In one embodiment, R¹ is hydrogen.

In one embodiment, R¹ is CN.

In one embodiment, R¹ is C₁-C₆ alkyl.

In one embodiment, R² is hydrogen.

In one embodiment, R² is halo. In one embodiment, R² is F. In one embodiment, R² is Cl. In one embodiment, R² is Br. In one embodiment, R² is I.

In one embodiment, R² is C₁-C₆ alkyl.

In one embodiment, R³ is hydrogen.

In one embodiment, R³ is halo. In one embodiment, R³ is F. In one embodiment, R³ is Cl. In one embodiment, R³ is Br. In one embodiment, R³ is I.

In one embodiment, each of X¹, X², X³, and X⁴ is CH.

In one embodiment, X¹, X², and X³ are each CH, and X⁴ is N.

In one embodiment, X¹, X², and X⁴ are each CH, and X³ is N.

In one embodiment, X¹, X³, and X⁴ are each CH, and X² is N.

In one embodiment, X², X³, and X⁴ are each CH, and X¹ is N.

In one embodiment, X¹ and X² are each CH, and X³ and X⁴ are each N.

In one embodiment, X¹ and X³ are each CH, and X² and X⁴ are each N.

In one embodiment, X¹ and X⁴ are each CH, and X² and X³ are each N.

In one embodiment, X² and X³ are each CH, and X¹ and X⁴ are each N.

In one embodiment, X² and X⁴ are each CH, and X¹ and X³ are each N.

In one embodiment, X³ and X⁴ are each CH, and X¹ and X² are each N.

In one embodiment, n is 0.

In one embodiment, n is 1.

In one embodiment, the compound of Formula (I) is

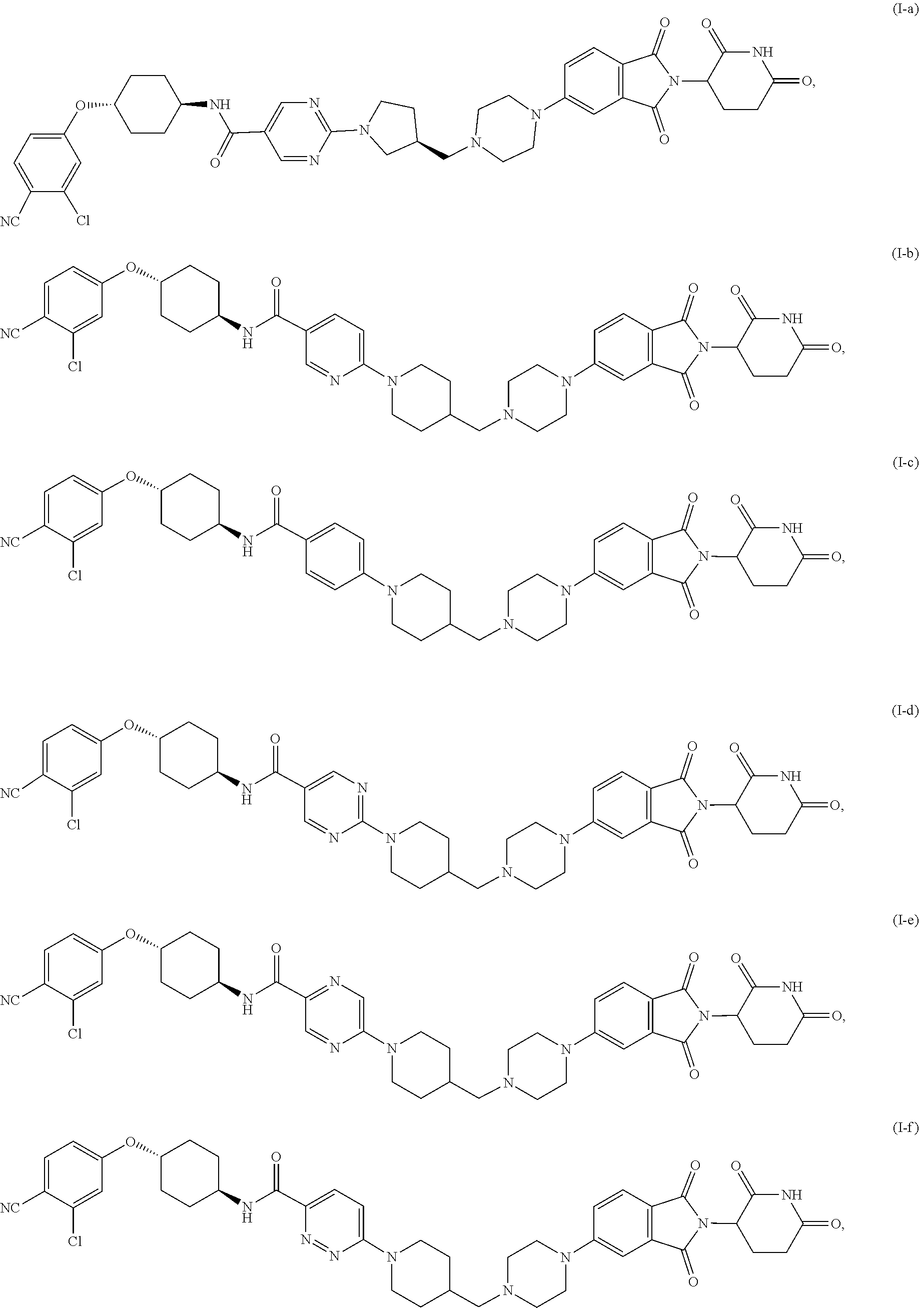

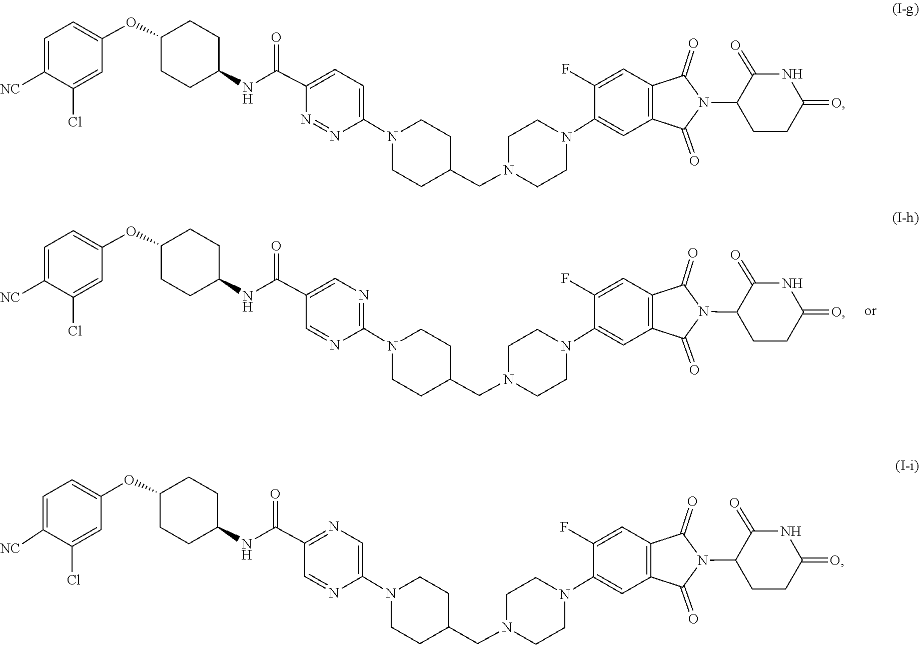 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "or" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

or a pharmaceutically acceptable salt, enantiomer, stereoisomer, solvate, polymorph, isotopic derivative, or prodrug thereof.

In one embodiment, the compound of Formula (I) is the compound of Formula (I-a):

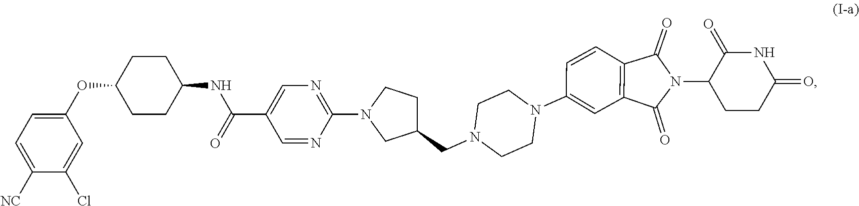 [SMILES: N#Cc1ccc(O[C@H]2CC[C@H](NC(=O)c3cnc(N4CC[C@@H](CN5CCN(c6ccc7c(c6)C(=O)N(C6CCC(=O)NC6=O)C7=O)CC5)C4)nc3)CC2)cc1Cl]

or a pharmaceutically acceptable salt, enantiomer, stereoisomer, solvate, polymorph, isotopic derivative, or prodrug thereof.

In one embodiment, the compound of Formula (I) is the compound of Formula (I-b):

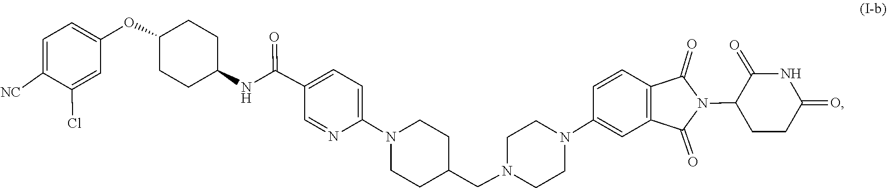 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)nc3)CC2)cc1Cl]

or a pharmaceutically acceptable salt, enantiomer, stereoisomer, solvate, polymorph, isotopic derivative, or prodrug thereof.

In one embodiment, the compound of Formula (I) is the compound of Formula (I-c):

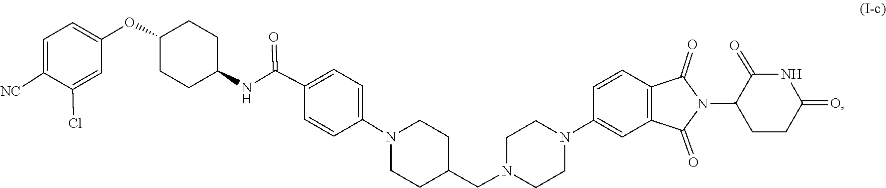 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)cc3)CC2)cc1Cl]

or a pharmaceutically acceptable salt, enantiomer, stereoisomer, solvate, polymorph, isotopic derivative, or prodrug thereof.

In one embodiment, the compound of Formula (I) is the compound of Formula (I-d):

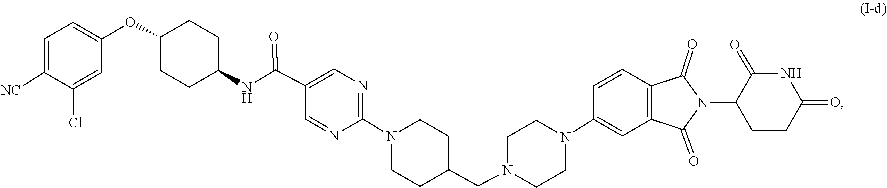 [SMILES: N#Cc1ccc(O[C@H]2CC[C@H](NC(=O)c3cnc(N4CCC(CN5CCN(c6ccc7c(c6)C(=O)N(C6CCC(=O)NC6=O)C7=O)CC5)CC4)nc3)CC2)cc1Cl]

or a pharmaceutically acceptable salt, enantiomer, stereoisomer, solvate, polymorph, isotopic derivative, or prodrug thereof.

In one embodiment, the compound of Formula (I) is the compound of Formula (I-e):

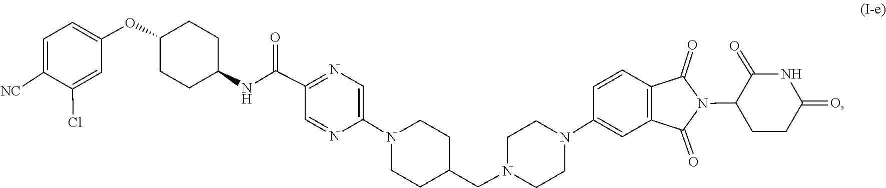 [SMILES: N#Cc1ccc(O[C@H]2CC[C@H](NC(=O)c3cnc(N4CCC(CN5CCN(c6ccc7c(c6)C(=O)N(C6CCC(=O)NC6=O)C7=O)CC5)CC4)cn3)CC2)cc1Cl]

or a pharmaceutically acceptable salt, enantiomer, stereoisomer, solvate, polymorph, isotopic derivative, or prodrug thereof.

In one embodiment, the compound of Formula (I) is the compound of Formula (I-f):

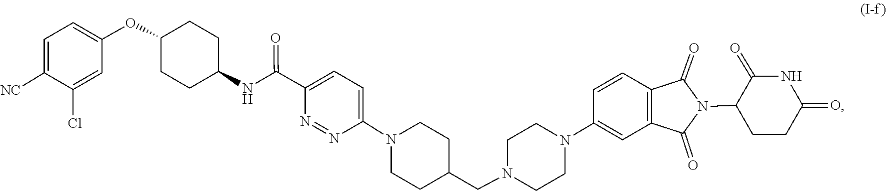 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)nn3)CC2)cc1Cl]

or a pharmaceutically acceptable salt, enantiomer, stereoisomer, solvate, polymorph, isotopic derivative, or prodrug thereof.

In one embodiment, the compound of Formula (I) is the compound of Formula (I-g):

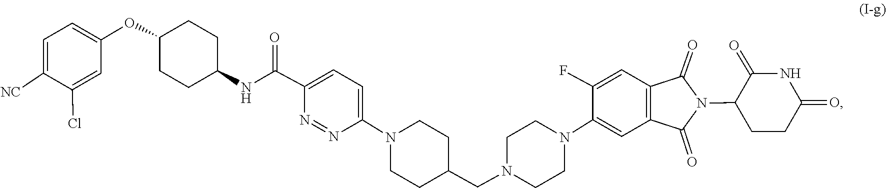 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt, enantiomer, stereoisomer, solvate, polymorph, isotopic derivative, or prodrug thereof.

In one embodiment, the compound of Formula (I) is the compound of Formula (I-h):

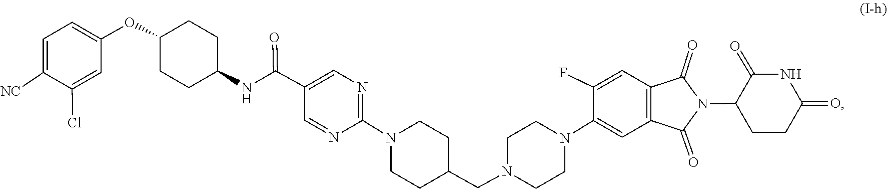 [SMILES: N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl]

or a pharmaceutically acceptable salt, enantiomer, stereoisomer, solvate, polymorph, isotopic derivative, or prodrug thereof.

In one embodiment, the compound of Formula (I) is the compound of Formula (I-i):

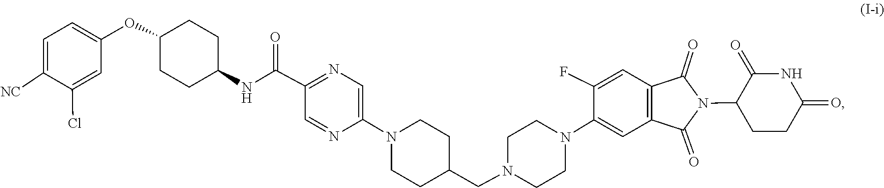 [SMILES: N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

or a pharmaceutically acceptable salt, enantiomer, stereoisomer, solvate, polymorph, isotopic derivative, or prodrug thereof.

A compound of Formula (I) may be synthesized using standard synthetic methods and procedures for the preparation of organic molecules and functional group transformations and manipulations, including the use of protective groups, as can be obtained from the relevant scientific literature or from standard reference textbooks in the field. Although not limited to any one or several sources, recognized reference textbooks of organic synthesis include: Smith, M. B.; March, J. March's Advanced Organic Chemistry: Reactions, Mechanisms, and Structure, 5^{th} ed.; John Wiley & Sons: New York, 2001; and Greene, T. W.; Wuts, P. G. M. Protective Groups in Organic Synthesis, 3^{rd}; John Wiley & Sons: New York, 1999. Methods for preparing a compound of Formula (I) are described in U.S. Patent Application Publication No. 2018/0099940, now U.S. Pat. No. 10,584,101, and in U.S. patent application Ser. No. 17/313,679. The contents of each of which are incorporated herein in their entireties.

### Methods of Ubiquitinating/Degrading a Target Protein in a Cell

The present invention provides a method of ubiquitinating and degrading a target protein in a cell. The method comprises administering a bifunctional composition comprising an E3 ubiquitin ligase binding moiety and a protein targeting moiety, preferably linked through a linker moiety, as otherwise described herein, wherein the E3 ubiquitin ligase binding moiety is coupled to the protein targeting moiety and wherein the E3 ubiquitin ligase binding moiety recognizes a ubiquitin pathway protein (e.g., an ubiquitin ligase, preferably an E3 ubiquitin ligase such as cereblon) and the protein targeting moiety recognizes the target protein such that ubiquitination of the target protein will occur when the target protein is placed in proximity to the ubiquitin ligase, thereby triggering degradation of the target protein via the proteasome system and the control of protein levels. The control of protein levels afforded by the present invention provides treatment of a disease state, symptom or condition, which is modulated through the target protein by lowering the level of that protein in the cells of a patient.

In one embodiment, the present invention is directed to a method of treating a patient in need thereof for a disease state, symptom or condition modulated through the targeted protein where the degradation of that protein will produce a therapeutic effect in that patient, the method comprising administering to a patient in need thereof an effective amount of a compound according to the present invention, optionally in combination with another bioactive agent (e.g., abiraterone). The disease state, symptom or condition may be caused by overexpression of the targeted protein, which leads to a disease state, symptom and/or condition.

### Methods of Treatment

In one aspect, the present application pertains to a method of treating and/or preventing cancer comprising administering to a subject in need thereof a therapeutically effective amount of a compound of Formula (I), or a pharmaceutically acceptable salt, enantiomer, stereoisomer, solvate, polymorph, isotopic derivative, or prodrug thereof.

The methods of treating cancer described herein preferably result in a cessation or slowing of tumor growth, and more preferably a reduction in tumor size. Alternatively, or in addition, the cancer is metastatic cancer and this method of treatment includes inhibition of metastatic cancer cell invasion.

In one embodiment, the cancer is prostate cancer.

In one embodiment, the cancer is metastatic prostate cancer.

In one embodiment, the cancer is castrate-resistant prostate cancer.

In one embodiment, the cancer is metastatic, castrate-resistant prostate cancer.

In one aspect, the application pertains to treating prostate cancer with a compound of Formula (I), wherein the compound of Formula (I) refers to a compound with the following structure:

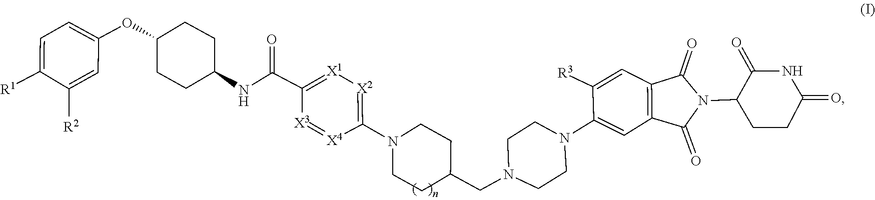

or a pharmaceutically acceptable salt, enantiomer, stereoisomer, solvate, polymorph, isotopic derivative, or prodrug thereof, wherein R¹, R², R³, X¹, X², X³, and X⁴ and n are defined herein. In one embodiment, the cancer is metastatic prostate cancer. In one embodiment, the cancer is castrate-resistant or castration-resistant prostate cancer. In one embodiment, the cancer is metastatic, castrate-resistant prostate cancer.

In one aspect, the application pertains to treating prostate cancer with a compound of Formula (I), wherein the compound of Formula (I) is selected from the group consisting of:

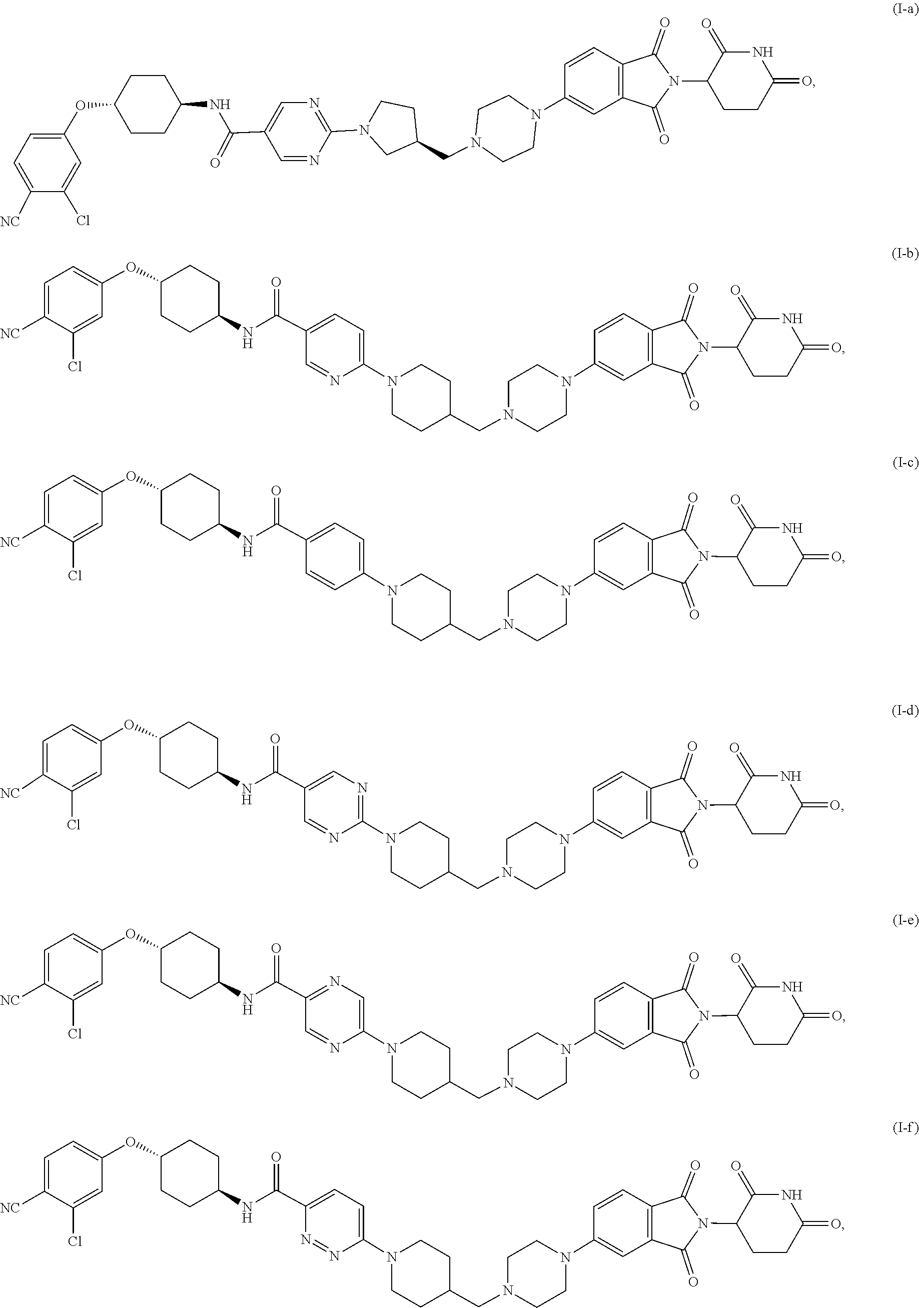

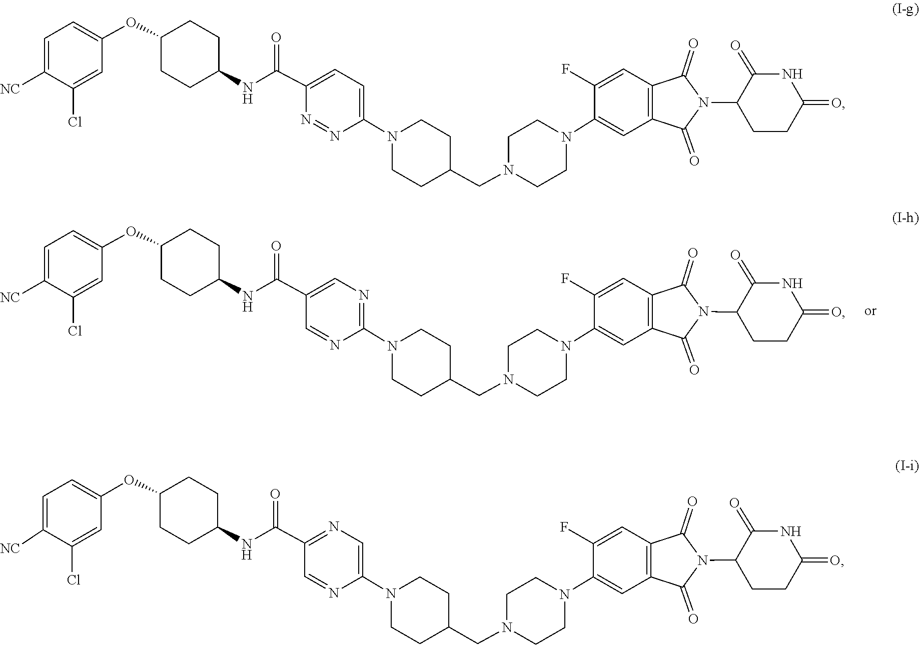 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "or" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

or a pharmaceutically acceptable salt, enantiomer, stereoisomer, solvate, polymorph, isotopic derivative, or prodrug thereof. In one embodiment, the cancer is metastatic prostate cancer. In one embodiment, the prostate cancer is castrate-resistant or castration-resistant prostate cancer. In one embodiment, the prostate cancer is metastatic, castrate-resistant prostate cancer.

In one aspect, the application pertains to treating prostate cancer with a compound of Formula (I) in combination with another bioactive agent, wherein the compound of Formula (I) refers to a compound with the following structure:

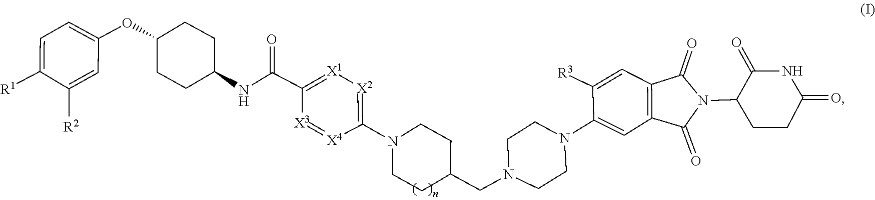

or a pharmaceutically acceptable salt, enantiomer, stereoisomer, solvate, polymorph, isotopic derivative, or prodrug thereof, wherein R¹, R², R³, X¹, X², X³, and X⁴ and n are defined herein. In one embodiment, the compound of Formula (I) is the compound of Formula (I-g).

In one embodiment, the prostate cancer treated with the combination of a compound of Formula (I) and another bioactive agent is metastatic prostate cancer. In one embodiment, the prostate cancer treated with the combination of a compound of Formula (I) and another bioactive agent is castrate-resistant or castration-resistant prostate cancer. In one embodiment, the prostate cancer treated with the combination of a compound of Formula (I) and another bioactive agent is metastatic, castrate-resistant prostate cancer. In one embodiment, the other bioactive agent is abiraterone or a pharmaceutically acceptable salt thereof. In one embodiment, the other bioactive agent is abiraterone acetate.

In one aspect, treating cancer results in a reduction in size of a tumor. A reduction in size of a tumor may also be referred to as “tumor regression.” Preferably, after treatment, tumor size is reduced by about 5% to about 40% relative to its size prior to treatment; more preferably, tumor size is reduced by about 10% to about 50%; more preferably, reduced by about 20% to about 60%; more preferably, reduced by about 30% to about 70%; more preferably, reduced by about 40% to about 80%; more preferably, reduced by about 50% to about 90%; and most preferably, reduced by about 75% to about 95%. Size of a tumor may be measured by any reproducible means of measurement. In a preferred aspect, size of a tumor may be measured as a diameter of the tumor.

In another aspect, treating cancer results in a reduction in tumor volume. Preferably, after treatment, tumor volume is reduced by about 5% to about 40% relative to its size prior to treatment; more preferably, tumor volume is reduced by about 10% to about 50%; more preferably, reduced by about 20% to about 60%; more preferably, reduced by about 30% to about 70%; more preferably, reduced by about 40% to about 80%; more preferably, reduced by about 50% to about 90%; and more preferably, reduced by about 75% to about 95%. Tumor volume may be measured by any reproducible means of measurement.

In another aspect, treating cancer results in a decrease in number of tumors. Preferably, after treatment, tumor number is reduced by about 5% to about 40% relative to number prior to treatment; more preferably, tumor number is reduced by about 10% to about 50%; more preferably, reduced by about 20% to about 60%; more preferably, reduced by about 30% to about 70%; more preferably, reduced by about 40% to about 80%; more preferably, reduced by about 50% to about 90%; and more preferably, reduced by about 75% to about 90%. Number of tumors may be measured by any reproducible means of measurement. In a preferred aspect, number of tumors may be measured by counting tumors visible to the naked eye or at a specified magnification. In a preferred aspect, the specified magnification is 2×, 3×, 4×, 5×, 10×, or 50×.

In another aspect, treating cancer results in a decrease in number of metastatic lesions in other tissues or organs distant from the primary tumor site. Preferably, after treatment, the number of metastatic lesions is reduced by about 5% to about 40% relative to number prior to treatment; more preferably, the number of metastatic lesions is reduced by about 10% to about 50%; more preferably, reduced by about 20% to about 60%; more preferably, reduced by about 30% to about 70%; more preferably, reduced by about 40% to about 80%; more preferably, reduced by about 50% to about 90%; and preferably, reduced by about 75% to about 95%. The number of metastatic lesions may be measured by any reproducible means of measurement. In a preferred aspect, the number of metastatic lesions may be measured by counting metastatic lesions visible to the naked eye or at a specified magnification. In a preferred aspect, the specified magnification is 2×, 3×, 4×, 5×, 10×, or 50×.

In another aspect, treating cancer results in an increase in average survival time of a population of treated subjects in comparison to a population receiving carrier alone. Preferably, the average survival time is increased by more than about 30 days; more preferably, by more than about 60 days; more preferably, by more than about 90 days; and most preferably, by more than about 120 days. An increase in average survival time of a population may be measured by any reproducible means. In a preferred aspect, an increase in average survival time of a population may be measured, for example, by calculating for a population the average length of survival following initiation of treatment with an active agent or compound. In another preferred aspect, an increase in average survival time of a population may also be measured, for example, by calculating for a population the average length of survival following completion of a first round of treatment with an active agent or compound.

In another aspect, treating cancer results in an increase in average survival time of a population of treated subjects in comparison to a population of untreated subjects. Preferably, the average survival time is increased by more than about 30 days; more preferably, by more than about 60 days; more preferably, by more than about 90 days; and most preferably, by more than about 120 days. An increase in average survival time of a population may be measured by any reproducible means. In a preferred aspect, an increase in average survival time of a population may be measured, for example, by calculating for a population the average length of survival following initiation of treatment with an active agent or compound. In another preferred aspect, an increase in average survival time of a population may also be measured, for example, by calculating for a population the average length of survival following completion of a first round of treatment with a compound of Formula (I).

In another aspect, treating cancer results in a decrease in tumor growth rate. Preferably, after treatment, tumor growth rate is reduced by at least about 5% relative to the growth rate prior to treatment; more preferably, tumor growth rate is reduced by at least about 10%; more preferably, reduced by at least about 20%; more preferably, reduced by at least about 30%; more preferably, reduced by at least about 40%; more preferably, reduced by at least about 50%; even more preferably, reduced by at least about 50%; and most preferably, reduced by at least about 75%. Tumor growth rate may be measured by any reproducible means of measurement. In a preferred aspect, tumor growth rate is measured according to a change in tumor diameter per unit time.

In another aspect, treating cancer results in a decrease in tumor regrowth. Preferably, after treatment, tumor regrowth is less than about 5%; more preferably, tumor regrowth is less than about 10%; more preferably, less than about 20%; more preferably, less than about 30%; more preferably, less than about 40%; more preferably, less than about 50%; more preferably, less than about 50%; and most preferably, less than about 75%. Tumor regrowth may be measured by any reproducible means of measurement. In a preferred aspect, tumor regrowth is measured, for example, by measuring an increase in the diameter of a tumor after a prior tumor shrinkage that followed treatment. In another preferred aspect, a decrease in tumor regrowth is indicated by failure of tumors to reoccur after treatment has stopped.

In certain embodiments, patients should not initiate, continue, or maintain the same level of, treatment with the BCRP substrate rosuvastatin while being treated with Compound (I-g).

In certain embodiments, the BCRP substrate rosuvastatin should be discontinued or reduced at least 7 days, at least 6 days, at least 5 days, at least 4 days, at least 3 days, at least 2 days, at least 1 day, or at least 7 half-lives, at least 6 half-lives, at least 5 half-lives, at least 4 half-lives, at least 3 half-lives, at least 2 half-lives, or at least 1 half-life prior to starting treatment with Compound (I-g).

In certain embodiments, the present invention provides packaged dosage forms of Compound (I-g) with printed instructions to avoid, reduce or discontinue administering rosuvastatin prior to administering Compound (I-g).

The dosages of a compound of Formula (I) for any of the methods and uses described herein vary depending on the agent, the age, weight, and clinical condition of the recipient subject, and the experience and judgment of the clinician or practitioner administering the therapy, among other factors affecting the selected dosage.

The therapeutically effective amount of a compound of Formula (I) may be administered one or more times over a day for up to 30 or more days, followed by 1 or more days of non-administration of a compound of Formula (I). This type of treatment schedule, i.e., administration of a compound of Formula (I) on consecutive days followed by non-administration of a compound of Formula (I) on consecutive days may be referred to as a treatment cycle. A treatment cycle may be repeated as many times as necessary to achieve the intended affect.

In one embodiment, the therapeutically effective amount of a compound of Formula (I) is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, thirty consecutive days, or, once, twice, three times, four times, or more daily, in single or divided doses, for 2 months, 3 months, 4 months, 5 months, 6 months, or longer.

In one embodiment, the therapeutically effective amount of a compound of Formula (I) is about 10 to about 40 mg, about 20 to about 50 mg, about 30 to about 60 mg, about 40 to about 70 mg, about 50 to about 80 mg, about 60 to about 90 mg, about 70 to about 100 mg, about 80 to about 110 mg, about 90 to about 120 mg, about 100 to about 130 mg, about 110 to about 140 mg, about 120 to about 150 mg, about 130 to about 160 mg, about 140 to about 170 mg, about 150 to about 180 mg, about 160 to about 190 mg, about 170 to about 200 mg, about 180 to about 210 mg, about 190 to about 220 mg, about 200 to about 230 mg, about 210 to about 240 mg, about 220 to about 250 mg, about 230 to about 260 mg, about 240 to about 270 mg, about 250 to about 280 mg, about 260 to about 290 mg, about 270 to about 300 mg, about 280 to about 310 mg, about 290 to about 320 mg, about 300 to about 330 mg, about 310 to about 340 mg, about 320 to about 350 mg, about 330 to about 360 mg, about 340 to about 370 mg, about 350 to about 380 mg, about 360 to about 390 mg, about 370 to about 400 mg, about 380 to about 410 mg, about 390 to about 420 mg, about 400 to about 430 mg, about 410 to about 440 mg, about 420 to about 450 mg, about 430 to about 460 mg, about 440 to about 470 mg, about 450 to about 480 mg, about 460 to about 490 mg, about 470 to about 500 mg, about 480 to about 510 mg, about 490 to about 520 mg, about 500 to about 530 mg, about 510 to about 540 mg, about 520 to about 550 mg, about 530 to about 560 mg, about 540 to about 570 mg, about 550 to about 580 mg, about 560 to about 590 mg, about 570 to about 600 mg, about 580 to about 610 mg, about 590 to about 620 mg, about 600 to about 630 mg, about 610 to about 640 mg, about 620 to about 650 mg, about 630 to about 660 mg, about 640 to about 670 mg, about 650 to about 680 mg, about 660 to about 690 mg, about 670 to about 700 mg, about 680 to about 710 mg, about 690 to about 720 mg, about 700 to about 730 mg, about 710 to about 740 mg, about 720 to about 750 mg, about 730 to about 760 mg, about 740 to about 770 mg, about 750 to about 780 mg, about 760 to about 790 mg, about 770 to about 800 mg, about 780 to about 810 mg, about 790 to about 820 mg, about 800 to about 830 mg, about 810 to about 840 mg, about 820 to about 850 mg, about 830 to about 860 mg, about 840 to about 870 mg, about 850 to about 880 mg, about 860 to about 890 mg, about 870 to about 900 mg, about 880 to about 910 mg, about 890 to about 920 mg, about 900 to about 930 mg, about 910 to about 940 mg, about 920 to about 950 mg, about 930 to about 960 mg, about 940 to about 970 mg, about 950 to about 980 mg, about 960 to about 990 mg, or about 970 to about 1,000 mg administered once, twice, three times, four times, or more daily in single or divided doses (which dose may be adjusted for the patient's weight in kg, body surface area in m², and age in years).

In one embodiment, the therapeutically effective amount of a compound of Formula (I) is about 70 mg to about 1000 mg administered once, twice, three times, four times, or more daily in single or divided doses (which dose may be adjusted for the patient's weight in kg, body surface area in m², and age in years).

In one embodiment, the therapeutically effective amount of a compound of Formula (I) is about 70 mg, 105 mg, 140 mg, 175 mg, 210 mg, 245 mg, 280 mg, 315 mg, 350 mg, 385 mg, 420 mg, 455 mg, 490 mg, 525 mg, 560 mg, 595 mg, 630 mg, 665 mg, or 700 mg administered once, twice, three times, four times, or more daily in single or divided doses (which dose may be adjusted for the patient's weight in kg, body surface area in m², and age in years).

The therapeutically effective amount of a compound of Formula (I) can also range from about 0.01 mg/kg per day to about 100 mg/kg per day. In an aspect, therapeutically effective amount of a compound of Formula (I) can range from about 0.05 mg/kg per day to about 10 mg/kg per day. In an aspect, therapeutically effective amount of a compound of Formula (I) can range from about 0.075 mg/kg per day to about 5 mg/kg per day. In an aspect, therapeutically effective amount of a compound of Formula (I) can range from about 0.10 mg/kg per day to about 1 mg/kg per day. In an aspect, therapeutically effective amount of a compound of Formula (I) can range from about 0.20 mg/kg per day to about 0.70 mg/kg per day.

In one embodiment, the therapeutically effective amount of a compound of Formula (I) is about 0.10 mg/kg per day, about 0.15 mg/kg per day, about 0.20 mg/kg per day, about 0.25 mg/kg per day, about 0.30 mg/kg per day, about 0.35 mg/kg per day, about 0.40 mg/kg per day, about 0.45 mg/kg per day, about 0.50 mg/kg per day, about 0.55 mg/kg per day, about 0.60 mg/kg per day, about 0.65 mg/kg per day, about 0.70 mg/kg per day, about 0.75 mg/kg per day, about 0.80 mg/kg per day, about 0.85 mg/kg per day, about 0.90 mg/kg per day, about 0.95 mg/kg per day, or about 1.00 mg/kg per day.

In one embodiment, the therapeutically effective amount of a compound of Formula (I) is about 1.05 mg/kg per day, about 1.10 mg/kg per day, about 1.15 mg/kg per day, about 1.20 mg/kg per day, about 1.25 mg/kg per day, about 1.30 mg/kg per day, about 1.35 mg/kg per day, about 1.40 mg/kg per day, about 1.45 mg/kg per day, about 1.50 mg/kg per day, about 1.55 mg/kg per day, about 1.60 mg/kg per day, about 1.65 mg/kg per day, about 1.70 mg/kg per day, about 1.75 mg/kg per day, about 1.80 mg/kg per day, about 1.85 mg/kg per day, about 1.90 mg/kg per day, about 1.95 mg/kg per day, or about 2.00 mg/kg per day.

In one embodiment, the therapeutically effective amount of a compound of Formula (I) is about 2 mg/kg per day, about 2.5 mg/kg per day, about 3 mg/kg per day, about 3.5 mg/kg per day, about 4 mg/kg per day, about 4.5 mg/kg per day, about 5 mg/kg per day, about 5.5 mg/kg per day, about 6 mg/kg per day, about 6.5 mg/kg per day, about 7 mg/kg per day, about 7.5 mg/kg per day, about 8.0 mg/kg per day, about 8.5 mg/kg per day, about 9.0 mg/kg per day, about 9.5 mg/kg per day, or about 10 mg/kg per day.

In one embodiment, the therapeutically effective amount of a compound of Formula (I) is administered to the subject once daily. In one embodiment, this daily dose of a compound of Formula (I) may administered to the subject all at once. In one embodiment, this daily dose of a compound of Formula (I) may administered to the subject in two portions (a divided dose). In one embodiment, this daily dose of a compound of Formula (I) may administered to the subject in three portions. In one embodiment, this daily dose of a compound of Formula (I) may administered to the subject in four portions. In one embodiment, this daily dose of a compound of Formula (I) may administered to the subject in five or more portions. In one embodiment, these portions are administered to the subject at regular intervals throughout the day, for example, every 12 hours, every 8 hours, every 6 hours, every 5 hours, every 4 hours, etc.

In one embodiment, the therapeutically effective amount of the compound of Formula (I) results in a mean day 15 AUC₀₋₂₄ of greater than about 4,250 ng*hr/mL, about 4,300 ng*hr/mL, about 4,350 ng*hr/mL, about 4,400 ng*hr/mL, about 4,450 ng*hr/mL, about 4,500 ng*hr/mL, about 4,550 ng*hr/mL, about 4,600 ng*hr/mL, about 4,650 ng*hr/mL, about 4,700 ng*hr/mL, about 4,750 ng*hr/mL, about 4,800 ng*hr/mL, about 4,850 ng*hr/mL, about 4,900 ng*hr/mL, about 4,950 ng*hr/mL, about 5,000 ng*hr/mL, 5,050 ng*hr/mL, about 5,100 ng*hr/mL, about 5,150 ng*hr/mL, about 5,200 ng*hr/mL, about 5,250 ng*hr/mL, about 5,300 ng*hr/mL, about 5,350 ng*hr/mL, about 5,400 ng*hr/mL, about 5,450 ng*hr/mL, about 5,500 ng*hr/mL, about 5,550 ng*hr/mL, about 5,600 ng*hr/mL, about 5,650 ng*hr/mL, about 5,700 ng*hr/mL, about 5,750 ng*hr/mL, about 5,800 ng*hr/mL, about 5,850 ng*hr/mL, about 5,900 ng*hr/mL, 5,950 ng*hr/mL, or about 6,000 ng*hr/mL.

In one embodiment, the therapeutically effective amount of the compound of Formula (I) results in a mean day 15 Cₘₐₓ of greater than about 250 ng/mL, about 255 ng/mL, about 260 ng/mL, about 265 ng/mL, about 270 ng/mL, about 275 ng/mL, about 280 ng/mL, about 285 ng/mL, about 290 ng/mL, about 295 ng/mL, about 300 ng/mL, about 305 ng/mL, about 310 ng/mL, about 315 ng/mL, about 320 ng/mL, about 325 ng/mL, about 330 ng/mL, about 335 ng/mL, about 340 ng/mL, about 345 ng/mL, about 350 ng/mL, about 355 ng/mL, about 360 ng/mL, about 365 ng/mL, about 370 ng/mL, about 375 ng/mL, about 380 ng/mL, about 385 ng/mL, about 390 ng/mL, about 395 ng/mL, about 400 ng/mL, about 405 ng/mL, about 410 ng/mL, about 415 ng/mL, about 420 ng/mL, about 425 ng/mL, about 430 ng/mL, about 435 ng/mL, about 440 ng/mL, about 445 ng/mL, or about 450 ng/mL.

The therapeutically effective amount of a compound of Formula (I) can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of a compound of Formula (I) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

In one embodiment, for the methods of treating prostate cancer with the combination of a compound of Formula (I) and another bioactive agent, the therapeutically effective amount of a compound of Formula (I) is described herein, and the therapeutically effective amount of the other bioactive agent is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, thirty consecutive days, or, once, twice, three times, four times, or more daily, in single or divided doses, for 2 months, 3 months, 4 months, 5 months, 6 months, or longer. In one embodiment, the other bioactive agent is abiraterone or a pharmaceutically acceptable salt thereof. In one embodiment, the other bioactive agent is abiraterone acetate.

In one embodiment, for the methods of treating prostate cancer with the combination of a compound of Formula (I) and abiraterone, or a pharmaceutically acceptable salt thereof, the therapeutically effective amount of a compound of Formula (I) is described herein, and the therapeutically effective amount of abiraterone, or a pharmaceutically acceptable salt thereof, is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, thirty consecutive days, or, once, twice, three times, four times, or more daily, in single or divided doses, for 2 months, 3 months, 4 months, 5 months, 6 months, or longer. In one embodiment, the abiraterone is abiraterone acetate.

In one embodiment, for the methods of treating prostate cancer with the combination of a compound of Formula (I) and abiraterone acetate, the therapeutically effective amount of a compound of Formula (I) is described herein, and the therapeutically effective amount of abiraterone acetate is 1,000 mg administered orally once daily for one, two, three, four, five, six, seven, eight, nine, ten, eleven, twelve, thirteen, fourteen, fifteen, thirty, or more consecutive days, in single or divided doses. In one embodiment, the abiraterone acetate is administered in combination with 5 mg of prednisone administered orally, twice daily. In one embodiment, the combination of the compound of Formula (I) and abiraterone acetate is administered to the subject in need thereof in the fasted state. In one embodiment, the subject does not eat for at least two hours before, and at least one hour after, the administration of the combination of the compound of Formula (I) and abiraterone acetate.

In one embodiment, the compound of Formula (I) and abiraterone acetate are administered to the subject simultaneously. In one embodiment, the compound of Formula (I) and abiraterone acetate are administered to the subject sequentially.

In one embodiment, the compound of Formula (I) and abiraterone acetate are administered to the subject in temporal proximity.

In some embodiments, “temporal proximity” means that administration of compound of Formula (I) occurs within a time period before or after the administration of abiraterone acetate, such that the therapeutic effect of the compound of Formula (I) overlaps with the therapeutic effect of abiraterone acetate. In some embodiments, the therapeutic effect of the compound of Formula (I) completely overlaps with the therapeutic effect of abiraterone acetate. In some embodiments, “temporal proximity” means that administration of the compound of Formula (I) occurs within a time period before or after the administration of abiraterone acetate, such that there is a synergistic effect between the compound of Formula (I) and abiraterone acetate.

“Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

### Pharmaceutical Compositions

In one embodiment, a compound of Formula (I) is formulated for oral administration. For example, in one embodiment, a compound of Formula (I) is formulated as a tablet that comprises zero, one, two, or more of each of the following: emulsifier; surfactant, binder; disintegrant, glidant; and lubricant.

In one embodiment, the emulsifier is hypromellose.

In one embodiment, the surfactant is vitamin E polyethylene glycol succinate.

In one embodiment, the binder (also referred to herein as a filler) is selected from the group consisting of microcrystalline cellulose, lactose monohydrate, sucrose, glucose, and sorbitol.

In one embodiment, the disintegrant is croscarmellose sodium.

In one embodiment, the glidant refers to a substance used to promote powder flow by reducing interparticle cohesion. In one embodiment, in the dosage forms of the disclosure, the glidant is selected from the group consisting of silicon dioxide, silica colloidal anhydrous, starch, and talc.

In one embodiment, the lubricant refers to a substance that prevents ingredients from sticking and/or clumping together in the machines used in preparation of the dosage forms of the disclosure. In one embodiment, in the dosage forms of the disclosure, the lubricant is selected from the group consisting of magnesium stearate, sodium stearyl fumarate, stearic acid, and vegetable stearin.

The pharmaceutical compositions containing a compound of Formula (I) may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of a compound of Formula (I) into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating a compound of Formula (I) in the required amount in an appropriate solvent with one or a combination of ingredients enumerated above, as required, followed by filtered sterilization. Generally, dispersions are prepared by incorporating the active agent or compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, a compound of Formula (I) can be incorporated with excipients and used in the form of tablets, troches, or capsules. Oral compositions can also be prepared using a fluid carrier for use as a mouthwash, wherein the agent or compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the agents or compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives.

Transmucosal administration can be accomplished through the use of nasal sprays or suppositories, or buccal or sub-lingual dosage forms such as rapidly dissolving tablets or strips. For transdermal administration, the active agents or compounds are formulated into ointments, salves, gels, or creams as generally known in the art.

In one aspect, a compound of Formula (I) is prepared with pharmaceutically acceptable carriers that will protect the agent or compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active agent or compound calculated to produce the desired therapeutic effect in association with the required pharmaceutical carrier. The specification for the dosage unit forms of the application are dictated by and directly dependent on the unique characteristics of a compound of Formula (I) and the particular therapeutic effect to be achieved.

The pharmaceutical compositions can be included in a container, pack, or dispenser together with instructions for administration.

Illustrative modes of administration for a compound of Formula (I) includes systemic or local administration such as oral, nasal, parenteral, transdermal, subcutaneous, vaginal, buccal, rectal or topical administration modes. In one embodiment, the compound of Formula (I), or a pharmaceutically acceptable salt or hydrate thereof, is administered orally. In one embodiment, the compound of Formula (I) is administered as a tablet, capsule, caplet, solution, suspension, syrup, granule, bead, powder, or pellet.

Illustrative pharmaceutical compositions are tablets and gelatin capsules comprising a salt of compound of Formula (I) and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the salt such as cyclodextrin, hydroxypropyl-cyclodextrin, PEG400, and/or PEG200.

For preparing pharmaceutical compositions from a compound of Formula (I), or a salt or hydrate thereof, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. For example, water or water-propylene glycol solutions for parenteral injection or addition of sweeteners and opacifiers for oral solutions, suspensions and emulsions. Liquid form preparations may also include solutions for intranasal administration.

Liquid, particularly injectable, compositions can, for example, be prepared by dissolution, dispersion, etc. For example, the disclosed salt is dissolved in or mixed with a pharmaceutically acceptable solvent such as, for example, water, saline, aqueous dextrose, glycerol, ethanol, and the like, to thereby form an injectable isotonic solution or suspension.

Proteins such as albumin, chylomicron particles, or serum proteins can be used to solubilize the disclosed compounds.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Aerosol preparations suitable for inhalation may include solutions and solids in powder form, which may be in combination with a pharmaceutically acceptable carrier, such as an inert compressed gas, e.g., nitrogen.

Also included are solid form preparations that are intended to be converted, shortly before use, to liquid form preparations for either oral or parenteral administration. Such liquid forms include solutions, suspensions and emulsions.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Pharmaceutical compositions can be prepared according to conventional mixing, granulating or coating methods, respectively, and the present pharmaceutical compositions can contain from about 0.1% to about 99%, from about 5% to about 90%, or from about 1% to about 20% of the disclosed salt by weight or volume.

All amounts of any component of an oral dosage form described herein, e.g., a tablet, that are indicated based on % w/w refer to the total weight of the oral dosage form, unless otherwise indicated.

### EXAMPLES

The disclosure is further illustrated by the following examples, which are not to be construed as limiting this disclosure in scope or spirit to the specific procedures herein described.

It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1—In Vitro Studies with Compound (I-g)

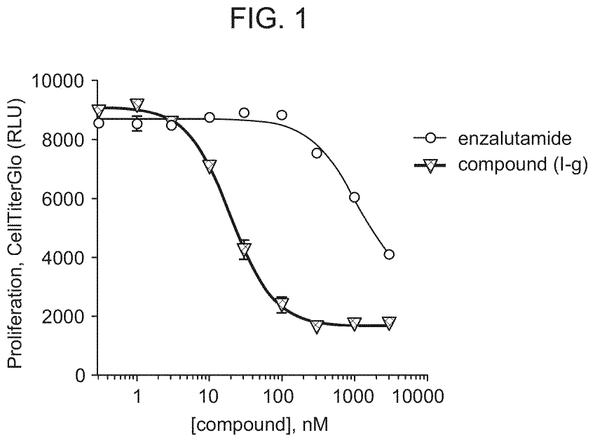

Compound (I-g) was shown to degrade 95% to 98% of androgen receptors (AR) in multiple cells lines typically used in prostate cancer research, including, for example, VCaP cells. (DC₅₀ in VCaP for Compound (I-g) is 1 nM.) Near-maximal degradation was observed within 4 hours of administration of Compound (I-g). Compound (I-g) inhibits VCaP proliferation about 60 times more potently than enzalutamide. (FIG. 1.)

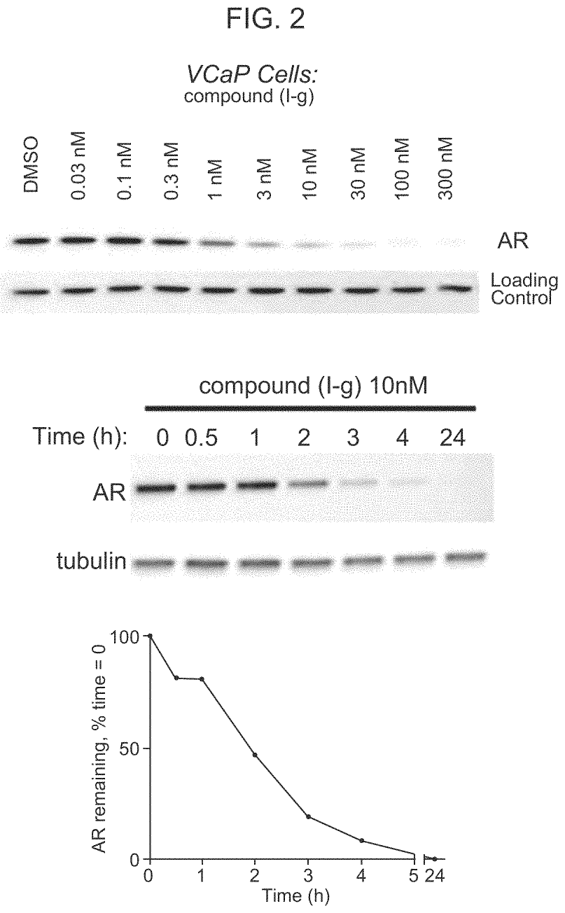

FIG. 2 shows the reduction of AR in VCaP tumor cells in response to treatment with Compound (I-g) at concentrations of 0.03 nM, 0.1 nM, 0.3 nM, 1 nM, 3 nM, 10 nM, 30 nM, 100 nM, and 300 nM.

### Example 2—In Vivo Studies with Animals and Assessment of the Preclinical Efficacious

Exposure Range for Compound (I-g)

Preclinical animal studies were performed with Compound (I-g) in VCaP xenograft animal models. VCaP was derived from a vertebral metastatic growth of a prostate carcinoma. It is a desirable cell line for in vivo studies as it exhibits many of the characteristics of clinical prostate carcinoma. VCaP is also a useful model to study AR resistance as it expresses AR splice variants that have been shown to drive resistance to AR antagonists. (European Urology. 2018 April; 73(4): 572-582.)

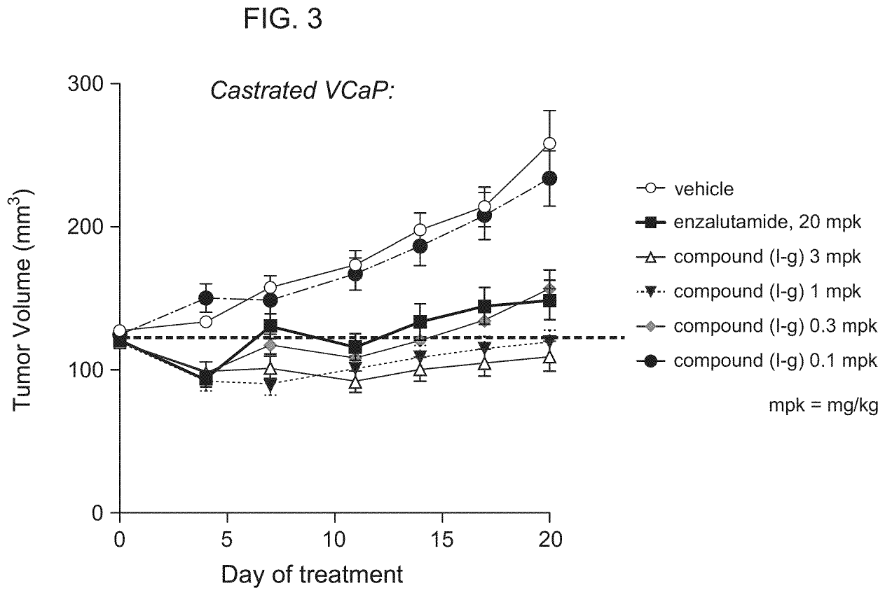

Oral, once daily administration of Compound (I-g) at doses of 0.1 mg/kg (mpk), 0.3 mg/kg, 1 mg/kg, and 3 mg/kg were performed in a castrated VCaP xenograft model (FIG. 3). Enzalutamide (20 mg/kg) and vehicle were also used as control groups.

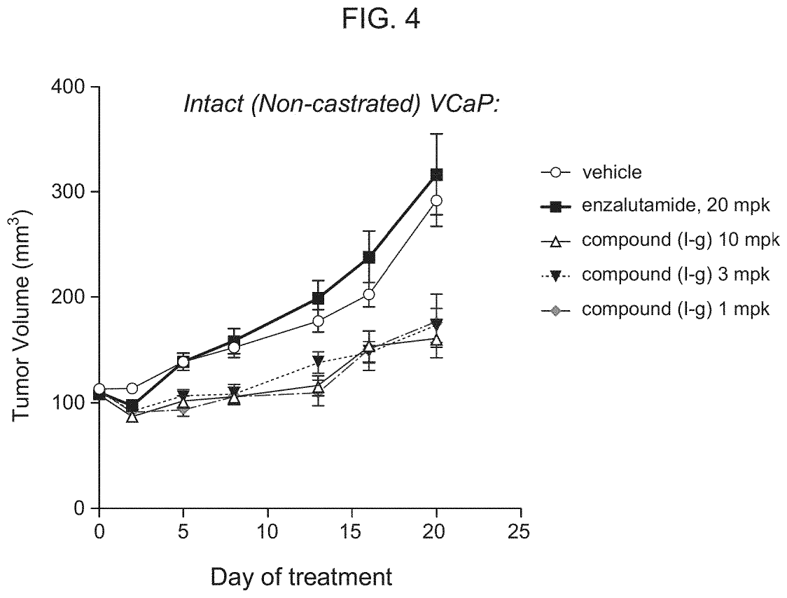

Oral, once daily administration of Compound (I-g) at doses of 1 mg/kg, 3 mg/kg, 10 mg/kg were performed in an intact (non-castrated) VCaP xenograft model (FIG. 4). Enzalutamide (20 mg/kg) and vehicle were also used as control groups.

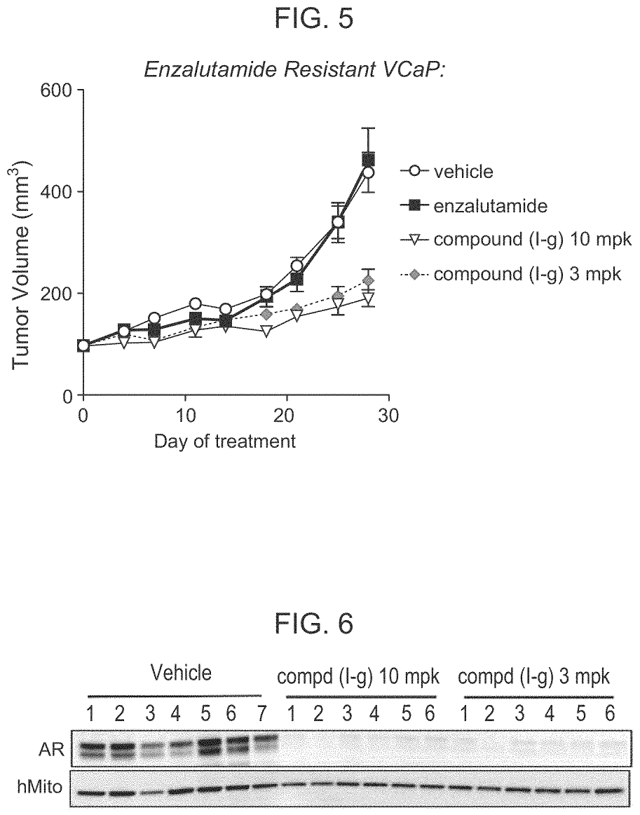

Oral, once daily administration of Compound (I-g) at doses of 3 mg/kg and 10 mg/kg were performed in an enzalutamide resistant VCaP xenograft model (FIG. 5). Enzalutamide (20 mg/kg) and vehicle were also used as control groups.

The pharmacokinetic results of oral, once daily administration of Compound (I-g) at doses of 1 mg/kg and 3 mg/kg are shown below in Table 1. A dose of 1 mg/kg of Compound (I-g) is the lowest dose that is superior to enzalutamide in a VCaP xenograft. A 3 mg/kg dose of Compound (I-g) was the lowest efficacious dose in an enzalutamide-resistant VCaP model (tumor growth inhibition of 70% compared to a control group).

FIG. 6 shows the reduction of AR in enzalutamide-resistant VCaP tumors in response to dosing with Compound (I-g) at 10 mg/kg and 3 mg/kg (oral, once daily).

TABLE 1

| Dose | Mean | Mean |
|---|---|---|
| (oral, once | AUC₀₋₂₄ | Cₘₐₓ |
| daily) | (ng*hr/mL)^{†} | (ng/mL)^{‡} |
| 1 mg/kg | 3628 | 224 |
| 3 mg/kg | 8106 | 507 |

Values represent total drug concentrations
^{†}AUC or Area Under the Curve is a measurement of total exposure
^{‡}Cₘₐₓ is a measurement of peak concentration during the dosing period

### Example 3—In Vivo Animal Studies with Compound (I-g) and Abiraterone

The combination of Compound (I-g) and abiraterone attenuated tumor growth more significantly than either agent alone in castrated VCaP xenografts.

### Example 4—Toxicology Studies

Animals were orally administered compound (I-g) once daily for 28 days, followed by a 14-day recovery for high-dose animals.

In dogs, once daily, oral doses of 3 mg/kg, 10 mg/kg, or 30 mg/kg of Compound (I-g) were administered. It was determined that the 30 mg/kg dose exceeded the maximum tolerated dose. Gastrointestinal alterations were observed at all dose levels (including vehicle alone). Reversible liver function enzyme elevation, which is considered non-adverse, was observed in some mid- and high-dose animals. Male animals exhibited decreased prostate weights, which may be attributable to the pharmacology of Compound (I-g).

In rats, males were administered once daily, oral doses of Compound (I-g) at doses of 20 mg/kg, 60 mg/kg, or 120 mg/kg. Female rats were administered once daily, oral doses of Compound (I-g) at doses of 20 mg/kg, 40 mg/kg, or 120 mg/kg.

Overall, Compound (I-g) was well tolerated at all doses, with the exception of the 80 mg/kg female cohort. These rats lost body weight and consumed less food. All of the findings in male high-dose rats were fully reversible (liver hypertrophy, femur physis thickening). Male rats also exhibited decreased prostate weights, which may be attributable to the pharmacology of Compound (I-g).

### Example 5—Phase I Clinical Trial Study Design with Compound (I-g)

A Phase I Clinical Trial with Compound (I-g) was undertaken. A traditional 3+3 dose escalation design was implemented. Starting dose of Compound (I-g) was 35 mg administered orally, once daily with food. Dose increases were dependent upon toxicities.

The key criteria for this trial were: men with metastatic, castrate-resistant prostate cancer (mCRPC); at least two prior systemic therapies, at least one of which was abiraterone or enzalutamide; and disease progression on most recent therapy (for example, rising PSA or two or more new lesions upon bone scan).

The key objectives for this trial were obtaining the maximum tolerated dose of Compound (I-g) and the recommended Phase II trial dose. Additional objectives included assessing overall safety of Compound (I-g), pharmacokinetics, anti-tumor activity (for example, PSA, RECIST), and biomarkers, including, for example, AR degradation in CTCs and pre- vs. post-treatment biopsies (when available); AR (and other) gene mutations, amplifications in ctDNA; and AR-V7 in CTCs.

### Example 6—Phase I Pharmacokinetic Data—Oral Administration of Compound (I-g)

In a Phase I clinical trial, Compound (I-g) was administered orally at a dose of 35 mg/day, 70 mg/day, and 140 mg/day. It was observed that treatment with 140 mg/day dose of Compound (I-g) enters the preclinical efficacious range associated with tumor growth inhibition.

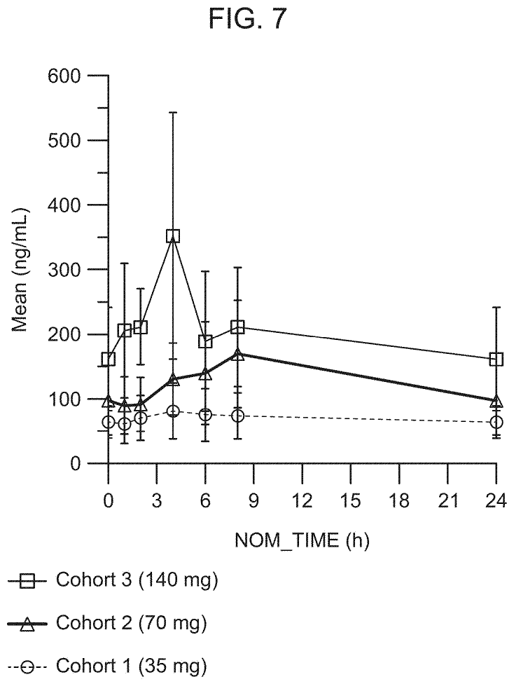

The initial pharmacokinetic results are shown below in Table 2, as well as in FIG. 7, which provides a representation of the mean concentrations of Compound (I-g) over a 24 hour time period after dosing on day 15 for all three tested doses (35 mg/day, 70 mg/day, and 140 mg/day).

TABLE 2

| Dose | Mean Day 1 | Mean Day 1 | Mean Day 15 | Mean Day 15 |
|---|---|---|---|---|
| (oral, once | AUC₀₋₂₄ | Cₘₐₓ | AUC₀₋₂₄ | Cₘₐₓ |
| daily) | (ng*hr/mL) | (ng/mL) | (ng*hr/mL)^{a} | (ng/mL) |
| 35 mg | 160.5 | 11.1 | 1701 | 83 |
| 70 mg | 300 | 19.6 | 2538 | 141 |
| 140 mg | 865 | 54 | 5023 | 353 |

^{a}Day 15 AUCs calculated using imputed 24 hours values.

### Example 7—Phase I Dose Escalation Studies with Compound (I-g)

Compound (I-g) was administered orally to human subjects at doses of 35 mg/day, 70 mg/day, 140 mg/day, and 280 mg/day. (n=3 for all dose groups, except the 70 mg/day cohort where n=4.)

In the 35 mg/day cohort, no dose limiting toxicity was observed. Also, no adverse events at grades 2, 3 or 4 were observed.

In the 70 mg/day cohort, no dose limiting toxicity has been observed. Also, no adverse events at grades 2, 3 or 4 have been observed, with the exception of grade 3 anemia that was unrelated to the administration of compound (I-g).

In the 140 mg/day cohort, no dose limiting toxicity has been observed. Also, no adverse events at grades 2, 3 or 4 have been observed. These results do not include one patient in this cohort group who was determined to be non-evaluable and treatment was discontinued on day 1.

### Example 8—Trial Design Modification to Discontinue Concomitant Medication

We have treated 18 patients with at least one dose of Compound (I-g) at once daily (QD) doses ranging from 35 mg to 280 mg. Overall, Compound (I-g) has been well tolerated. Of the 3 patients treated in the 280 mg QD cohort, one patient experienced dose limiting toxicity (DLT) of Grade 4 AST/ALT elevation followed by Grade 4 acute renal failure which required hemodialysis that was initiated after treatment was discontinued. Based on the available information, the renal event is suspected to be secondary to the hepatic event and may represent a case of hepatorenal syndrome. A second patient, who was enrolled at the 70 mg QD dose level, experienced a non-serious event of Grade 3 AST/ALT elevation during his 4^{th} cycle of therapy 28 days after a scheduled dose increase to 140 mg QD, a dose level that had been deemed safe by the Cohort Review Committee. At the time of AST/ALT elevation, both patients were taking rosuvastatin (Crestor®), a lipid lowering agent, associated with elevated liver enzyme levels and increased exposure in the setting of drug-drug interactions (AstraZeneca, 2018).

With regard to other statin agents, to date, no other patients in this study have received concomitant rosuvastatin. Five other patients were taking other statins (i.e., atorvastatin, pravastatin, simvastatin), as shown in Table 3. No other Grade 3 AST/ALT elevation was observed in those or any other patients that resulted in Compound (I-g) interruption or discontinuation.

TABLE 3
AST/ALT elevations observed with respect to other statins and concomitant
medications

| | Compound | Medication | AST/ALT | Treatment |
|---|---|---|---|---|
| Patient | (I-g) Dose | (dose) | elevation | outcome |
| 002-04- | 140 mg QD | atorvastatin | none | No change; Compound (I-g) held |
| 005 | | (20 mg QD) | | on C2D15 due to symptoms not |
| | | | | related to AST/ALT elevation |
| 004-02- | 140 mg | atorvastatin | Grade 1 | Continuing Compound (I-g) |
| 005 | QD* | (20 mg QD) | | without interruption but |
| | | | | discontinued atorvastatin due to |
| | | | | Grade 1 AST/ALT elevation; no |
| | | | | further liver enzyme elevations |
| | | | | have been reported to date |
| 003-04- | 280 mg QD | atorvastatin | none | No change up to discontinuation for |
| 004 | | (10 mg QD) | | disease progression |
| 004-01- | 35 mg QD | pravastatin | none | No change up to discontinuation for |
| 002 | | (40 mg QD) | | disease progression |
| 003-02- | 70 mg | simvastatin | none | No change |
| 001 | QD** | (40 mg QD) | | |
| 003-03- | 140 mg QD | amlodipine | Grade 1 | Patient taken off drug due to |
| 002 | | (10 mg QD) | | disease progression |

*Patient was dose-escalated from 70 mg QD.
** Patient was dose-escalated from 35 mg QD.
Source: Clinical database, as of 11-Feb-2020.

Only two other cases of AST/ALT elevation were found to be notable to date, both Grade 1 in severity. In one case, the patient was not taking any statin but was taking 10 mg QD amlodipine (Norvasc®). This Grade 1 AST/ALT elevation increased at the time of disease progression. In the other case, the patient was taking atorvastatin (20 mg QD) which was discontinued, but continued treatment with Compound (I-g) without interruption. No further liver enzyme elevations have been reported for this patient who continues on study.

A review of the 28-day Good Laboratory Practice (GLP) toxicology study in dogs reveals reversible elevations of AST and ALT at Compound (I-g) doses of 10 or 30 mg/kg/day. These elevations were found during scheduled assessments on study days 14 and 29. No correlated histopathology changes were observed. No evidence of renal impairment was observed in the 28-day GLP toxicology studies in either dogs or rats. Further, renal impairment is generally not attributed to available therapies that similarly target the androgen receptor (e.g., enzalutamide).

Literature indicates that rosuvastatin's potential as a substrate of DDI is likely due to inhibition of one or more drug transporters involved in the absorption and clearance of rosuvastatin, including BCRP, OATP1B1, OATP1B3, OAT3, and NTCP (ref). Compound (I-g) showed no inhibition against BCRP, OATP1B1, and OATP1B3 at 3 and 10 μM (due to solubility limitation in a preliminary study), suggesting IC₅₀>>10 μM for these 3 transporters which are implicated in the disposition of rosuvastatin. While the available data do not indicate inhibition by Compound (I-g) up to 10 μM in vitro, the limited concentration for the in vitro testing do not rule out the possibility of inhibition at much higher concentrations. In addition, an additive or greater biologic adverse effect of each agent independently at the hepatocyte level cannot be excluded.

A review of clinical and safety data, including laboratory test values and concomitant medications, showed that the 2 patients were both taking rosuvastatin. An exploratory, non-GLP method was developed to analyze for rosuvastatin and its major metabolite N-desmethyl rosuvastatin in retained pharmacokinetic plasma samples from the 2 patients.

One of 3 patients enrolled in the 280 mg QD cohort of Compound (I-g) was admitted to the hospital 16 days after initiating study therapy, with the reported SAEs of Grade 4 AST/ALT elevation followed by Grade 4 acute renal failure. There was no associated serum bilirubin elevation. The SAE was preceded by Grade 3 AST/ALT elevation noted on routine laboratory tests drawn 2 days earlier that prompted discontinuation of Compound (I-g), as well as discontinuation of rosuvastatin (20 mg QD) which the patient had been taking concomitantly. AST/ALT levels gradually decreased from 4901/4116 U/L to 280/83 U/L within 14 days after both Compound (I-g) and rosuvastatin were discontinued. Rosuvastatin levels obtained from the exploratory assay were found to increase concurrently with onset the AST/ALT elevations, suggesting that the AST/ALT elevation may have been related to a possible interaction between Compound (I-g) and rosuvastatin.

On Cycle 1 Day 8, the patient experienced a Grade 2 elevation in AST/ALT and continued on study as allowed per protocol. Patient returned for Cycle 1 Day 15 visit and a Grade 3 AST/ALT elevation was reported and renal function was normal. Treatment with Compound (I-g) was discontinued at this time. Four days after treatment discontinuation, acute renal impairment in this patient was noted, coinciding with the serious Grade 4 AST/ALT elevation. Renal injury subsequently required hemodialysis which began 1 day later. Based on the available information, the renal event is suspected to be secondary to the hepatic event and may represent a case of hepatorenal syndrome.

At the time of hospital discharge, the Grade 4 AST/ALT elevation had steadily decreased as indicated above, however the Grade 3 acute renal failure remained ongoing as the patient continued dialysis after discharge.

Another patient was dose-escalated in accordance with the protocol to 140 mg QD after 3 cycles at 70 mg QD, following safety clearance of the 140 mg dose level per protocol. A non-serious Grade 3 AST/ALT elevation occurred 28 days after dose escalation, prompting a dose interruption, and was resolved to Grade 1 within 5 days of dose interruption. It was noted that this patient was also taking rosuvastatin (40 mg QD). After re-challenge with 70 mg QD Compound (I-g) while continuing to take rosuvastatin, the patient had a second instance of Grade 3 AST/ALT elevation, which led to dose interruption of Compound (I-g) and discontinuation of rosuvastatin.

When this patient was re-challenged a second time with 70 mg QD ARV-110 following the discontinuation of rosuvastatin, no other AST/ALT elevation has been observed for 90 days. The observed increase and decrease of AST/ALT levels in this patient upon re-challenge with Compound (I-g) in the presence or absence of rosuvastatin further suggests that there may be a possible interaction between Compound (I-g) and rosuvastatin.

The following factors taken together lead to a conclusion that the 2 cases of significant AST/ALT elevation observed in protocol Compound (I-g)-mCRPC-101 may have been related to a possible drug-drug interaction between Compound (I-g) and rosuvastatin:

Of 22 patients treated to date in protocol Compound (I-g)-mCRPC-101, 1 patient experienced a Grade 4 AST/ALT elevation and a second patient experienced a Grade 3 AST/ALT elevation. Both were taking rosuvastatin concurrently with Compound (I-g).

Rosuvastatin plasma concentrations obtained from an exploratory, non-GLP assay increased concurrently with the increase in AST/ALT levels in both patients. Temporally, both cases of AST/ALT elevation began to resolve once one or both drugs were discontinued or interrupted.

Re-challenging with Compound (I-g) in the presence or absence of rosuvastatin in the patient with non-serious Grade 3 AST/ALT elevation further suggests a possible interaction between Compound (I-g) and rosuvastatin.

A review of AE and laboratory data conducted to investigate other cases of AST/ALT elevation was notable for only two other cases that were both Grade 1 in severity, one of which increased at the time of disease progression. Statins other than rosuvastatin (e.g., atorvastatin, pravastatin) have been taken concurrently with Compound (I-g) in 5 other patients.

No additional Grade 3 AST/ALT elevations were observed in those or any other patients that resulted in Compound (I-g) dose interruption or discontinuation.

Rosuvastatin is associated with AST/ALT elevations that may be dose dependent and has been shown to be a substrate of DDI with another AR targeted therapy, i.e., darolutamide (Bayer, 2019).

### REFERENCES

AstraZeneca. Crestor (Rosuvastatin Calcium) Tablets: Full Prescribing Information. (Reference ID: 4347984). Wilmington (Del.): AstraZeneca Pharmaceuticals LP; September 2018. Available at: https://www.accessdata.fda.gov/drugsatfda_docs/label/2018/021366s038lbl.pdf [Last accessed Jan. 28, 2020],
Bayer. Nubeqa (darolutamide) tablets, for oral use: Full Prescribing Information. (Reference ID: 4469847). Whippany (NJ): Bayer Healthcare Pharmaceuticals Inc.; July 2019. Available at: https://www.accessdata.fda.gov/drugsatfda_docs/label/2019/212099Orig1s000lbl.pdf [Last accessed Feb. 2, 2020],
Elsby R, et al, Understanding the Critical Disposition Pathways of Statins to Access Drug-Drug Interaction Risk During Drug Development: It's not Just About OATP1B1, Clin Pharmacol Ther, 92 (5), 584-98 November 2012.
FDA. In Vitro Drug Interaction Studies—Cytochrome P450 Enzyme and Transporter-Mediated Drug Interactions Guidance for Industry. January 2020.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

The methods of the disclosure have been described herein by reference to certain preferred embodiments. However, as particular variations thereon will become apparent to those skilled in the art, based on the disclosure set forth herein, the disclosure is not to be considered as limited thereto.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise.

It is to be understood that at least some of the descriptions of the disclosure have been simplified to focus on elements that are relevant for a clear understanding of the disclosure, while eliminating, for purposes of clarity, other elements that those of ordinary skill in the art will appreciate may also comprise a portion of the disclosure. However, because such elements are well known in the art, and because they do not necessarily facilitate a better understanding of the disclosure, a description of such elements is not provided herein.

Further, to the extent that a method does not rely on the particular order of steps set forth herein, the particular order of the steps recited in a claim should not be construed as a limitation on that claim.

All patents, patent applications, references and publications cited herein are fully and completely incorporated by reference as if set forth in their entirety. Such documents are not admitted to be prior art to the present disclosure.

### Embodiments

The methods of the present disclosure are further described with reference to the following numbered embodiments:

1. A method of treating prostate cancer in a subject in need thereof, comprising administering to the subject a therapeutically effective amount of a compound of Formula (I):

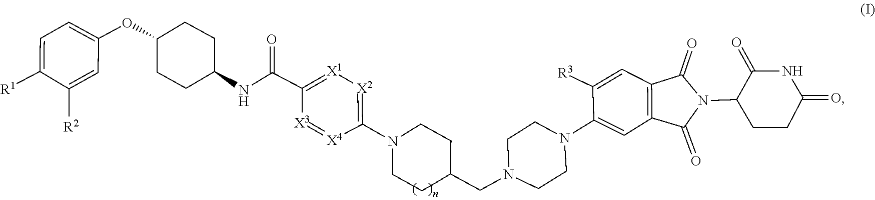

or a pharmaceutically acceptable salt, enantiomer, stereoisomer, solvate, polymorph, isotopic derivative, or prodrug thereof, wherein:

R¹ is hydrogen, CN, or C₁-C₆ alkyl;

R² is hydrogen, halo, or C₁-C₆ alkyl;

R³ is hydrogen or halo;

X¹ is CH or N;

X² is CH or N;

X³ is CH or N;

X⁴ is CH or N; and

n is 0 or 1;

provided that at least two of X¹, X², X³, and X⁴ are CH.

1A. A method of treating prostate cancer in a subject in need thereof, comprising administering to the subject a therapeutically effective amount of a compound of Formula (I):

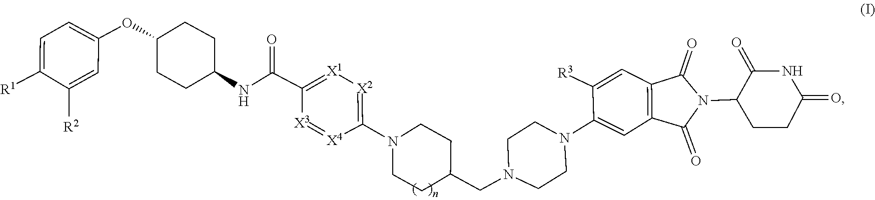

wherein:

R¹ is hydrogen, CN, or C₁-C₆ alkyl;

R² is hydrogen, halo, or C₁-C₆ alkyl;

R³ is hydrogen or halo;

X¹ is CH or N;

X² is CH or N;

X³ is CH or N;

X⁴ is CH or N; and

n is 0 or 1;

provided that at least two of X¹, X², X³, and X⁴ are CH.

2. The method of embodiment 1 or 1A, further comprising the step of discontinuing or reducing the administration of rosuvastatin to the subject prior to initiating administration of the compound of Formula (I).
3. The method of embodiment 1, 1A, or 2, wherein R¹ is hydrogen.
4. The method of embodiment 1, 1A, or 2, wherein R¹ is CN.
5. The method of embodiment 1, 1A, or 2, wherein R¹ is C₁-C₆ alkyl.
6. The method of any one of embodiments 1, 1A, or 2-5, wherein R² is hydrogen.
7. The method of any one of embodiments 1-5, wherein R² is halo.
8. The method of embodiment 7, wherein R² is F.
9. The method of embodiment 7, wherein R² is Cl.
10. The method of embodiment 7, wherein R² is Br.
11. The method of embodiment 7, wherein R² is I.
12. The method of any one of embodiments 1, 1A, or 2-5, wherein R² is C₁-C₆ alkyl.
13. The method of any one of embodiments 1, 1A, or 2-12, wherein R³ is hydrogen.
14. The method of any one of embodiments 1, 1A, or 2-12, wherein R³ is halo.
15. The method of embodiment 14, wherein R³ is F.
16. The method of embodiment 14, wherein R³ is Cl.
17. The method of embodiment 14, wherein R³ is Br.
18. The method of embodiment 14, wherein R³ is I.
19. The method of any one of embodiments 1, 1A, or 2-18, wherein each of X¹, X², X³, and X⁴ is CH.
20. The method of any one of embodiments 1, 1A, or 2-18, wherein X¹, X², and X³ are each CH, and X⁴ is N.
21. The method of any one of embodiments 1, 1A, or 2-18, wherein X¹, X², and X⁴ are each CH, and X³ is N.
22. The method of any one of embodiments 1, 1A, or 2-18, wherein X¹, X³, and X⁴ are each CH, and X² is N.
23. The method of any one of embodiments 1, 1A, or 2-18, wherein X², X³, and X⁴ are each CH, and X¹ is N.
24. The method of any one of embodiments 1, 1A, or 2-18, wherein X¹ and X² are each CH, and X³ and X⁴ are each N.
25. The method of any one of embodiments 1, 1A, or 2-18, wherein X¹ and X³ are each CH, and X² and X⁴ are each N.
26. The method of any one of embodiments 1, 1A, or 2-18, wherein X¹ and X⁴ are each CH, and X² and X³ are each N.
27. The method of any one of embodiments 1, 1A, or 2-18, wherein X² and X³ are each CH, and X¹ and X⁴ are each N.
28. The method of any one of embodiments 1, 1A, or 2-18, wherein X² and X⁴ are each CH, and X¹ and X³ are each N.
29. The method of any one of embodiments 1, 1A, or 2-18, wherein X³ and X⁴ are each CH, and X¹ and X² are each N.
30. The method of any one of embodiments 1, 1A, or 2-29, wherein n is 0.
31. The method of any one of embodiments 1, 1A, or 2-29, wherein nisi.
32. The method of any one of embodiments 1, 1A, or 2-31, wherein the prostate cancer is castrate-resistant prostate cancer.
33. The method of any one of embodiments 1, 1A, or 2-32, wherein the prostate cancer is metastatic prostate cancer.
34. The method of any one of embodiments 1, 1A, or 2-33, wherein the compound of Formula (I) is:

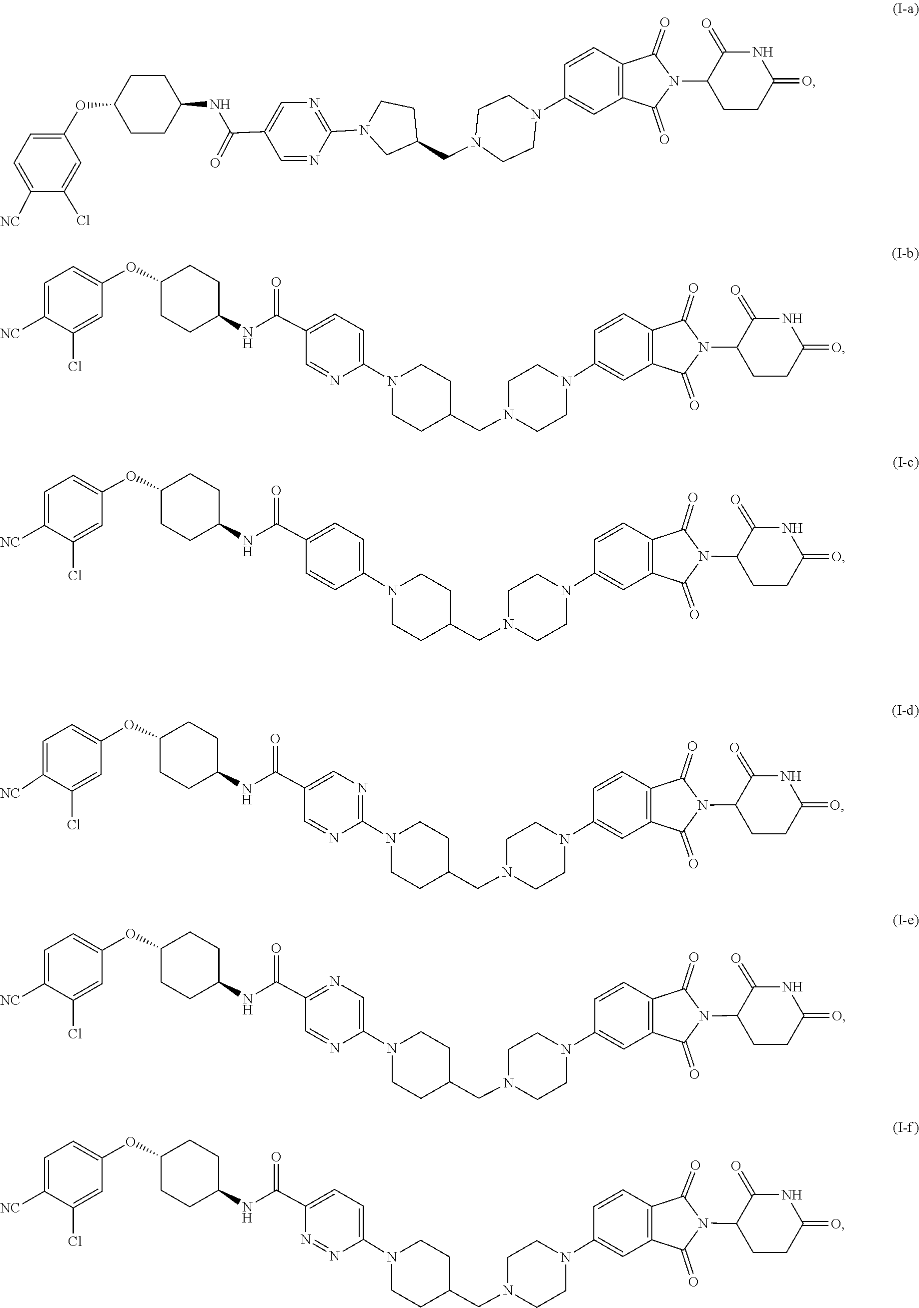

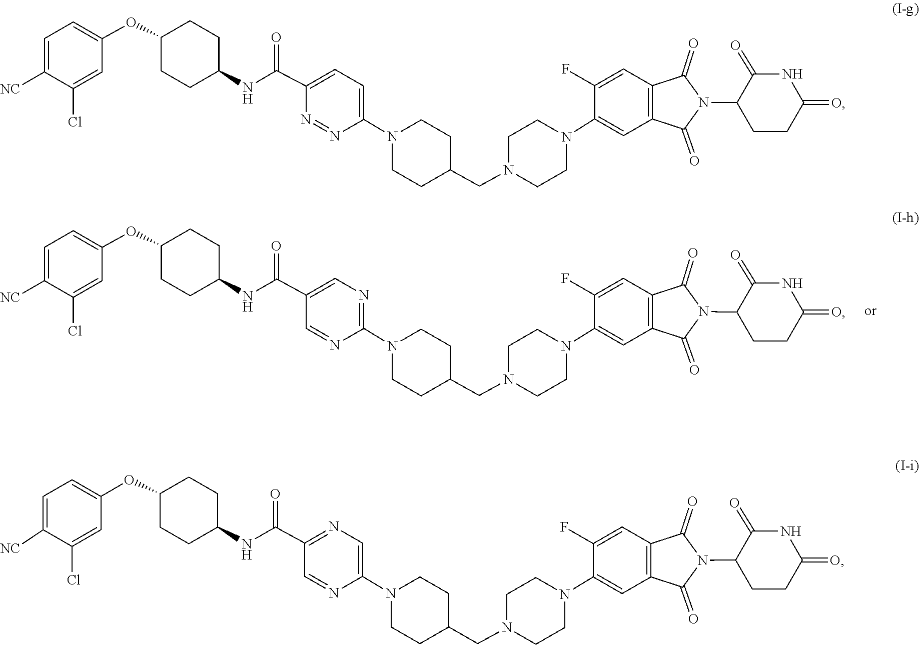 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "or" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

or a pharmaceutically acceptable salt, enantiomer, stereoisomer, solvate, polymorph, isotopic derivative, or prodrug thereof.
35. The method of any one of embodiments 1, 1A, or 2-34, wherein the compound of Formula (I) is administered orally to the subject.
36. The method of any one of embodiments 1, 1A, or 2-35, wherein the therapeutically effective amount of the compound of Formula (I) is administered to the subject once a day, twice a day, three times a day, or four times a day.
37. The method of any one of embodiments 1, 1A, or 2-36, wherein the therapeutically effective amount of the compound of Formula (I) is administered to the subject once a day.
38. The method of any one of embodiments 1, 1A, or 2-37, wherein the therapeutically effective amount of the compound of Formula (I) is administered to the subject all at once or is administered in two, three, or four portions.
39. The method of any one of embodiments 1, 1A, or 2-38, wherein the therapeutically effective amount of the compound of Formula (I) is about 70 mg to about 1000 mg.
40. The method of any one of embodiments 1, 1A, or 2-39, wherein the therapeutically effective amount of the compound of Formula (I) is about 100 mg to about 280 mg.
41. The method of any one of embodiments 1, 1A, or 2-40, wherein the therapeutically effective amount of the compound of Formula (I) results in a mean day 15 AUC₀₋₂₄ of greater than about 4,500 ng*hr/mL, about 4,600 ng*hr/mL, about 4,700 ng*hr/mL, about 4,800 ng*hr/mL, about 4,900 ng*hr/mL, about 5,000 ng*hr/mL, about 5,100 ng*hr/mL, about 5,200 ng*hr/mL, about 5,300 ng*hr/mL, 5,400 ng*hr/mL, about 5,500 ng*hr/mL, about 5,600 ng*hr/mL, about 5,700 ng*hr/mL, about 5,800 ng*hr/mL, about 5,900 ng*hr/mL, or about 6,000 ng*hr/mL.
42. The method of any one of embodiments 1, 1A, or 2-40, wherein the therapeutically effective amount of the compound of Formula (I) results in a mean day 15 AUC₀₋₂₄ of greater than about 4,500 ng*hr/mL and less than about 5,500 ng*hr/mL.
43. The method of any one of embodiments 1, 1A, or 2-42, wherein the therapeutically effective amount of the compound of Formula (I) results in a mean day 15 Cₘₐₓ of greater than about 300 ng/mL and less than about 400 ng/mL.
44. The method of any one of embodiments 1, 1A, or 2-43, wherein the therapeutically effective amount of the compound of Formula (I) results in a mean day 15 Cₘₐₓ of greater than about 330 ng/mL, about 335 ng/mL, about 340 ng/mL, about 345 ng/mL, about 350 ng/mL, about 355 ng/mL, about 360 ng/mL, about 365 ng/mL, about 370 ng/mL, about 375 ng/mL, or about 380 ng/mL.
45. The method of any one of embodiments 1, 1A, or 2-44, wherein the compound of Formula (I) is formulated as a tablet.
46. The method of embodiment 45, wherein the tablet comprises a compound of Formula (I) and, optionally, one or more of the following: emulsifier; surfactant; binder; disintegrant; glidant; and lubricant.
47. The method of any one of embodiments 1, 1A, or 2-46, wherein the subject is in a fed state.
48. The method of any one of embodiments 1, 1A, or 2-46, wherein the subject is in a fasted state.

1. A method of treating prostate cancer in a subject in need thereof, comprising administering to the subject a therapeutically effective amount of a compound of Formula (I), 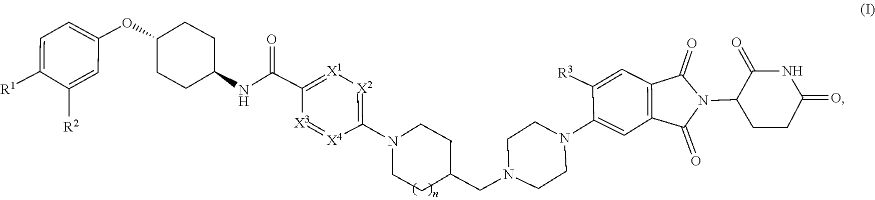
or a pharmaceutically acceptable salt, enantiomer, stereoisomer, solvate, polymorph, isotopic derivative, or prodrug thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH; and
further comprising the step of discontinuing or reducing the administration of rosuvastatin to the subject prior to initiating administration of the compound of Formula (I).

2. A method of treating prostate cancer in a subject in need thereof, comprising administering to the subject a therapeutically effective amount of a compound of Formula (I), 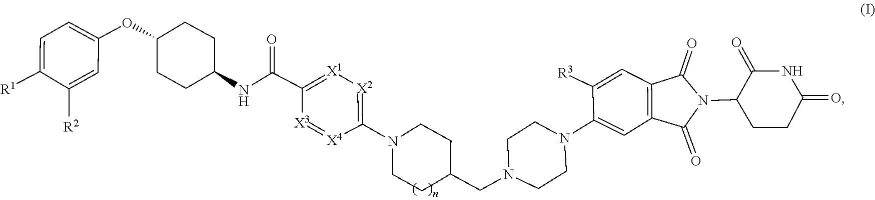
wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH; and
further comprising the step of discontinuing or reducing the administration of rosuvastatin to the subject prior to initiating administration of the compound of Formula (I).

3. The method of claim 1 or 2, wherein the prostate cancer is castrate-resistant prostate cancer.

4. The method of claim 1 or 2, wherein the prostate cancer is metastatic prostate cancer.

5. The method of claim 1 or 2, wherein R¹ is CN, R² is chloro, R³ is fluoro, n is 1, two of X¹, X², X³, and X⁴ are CH and the other two of X¹, X², X³, and X⁴ are N.

6-12. (canceled)

13. The method of claim 1 or 2, wherein the compound of Formula (I) is:

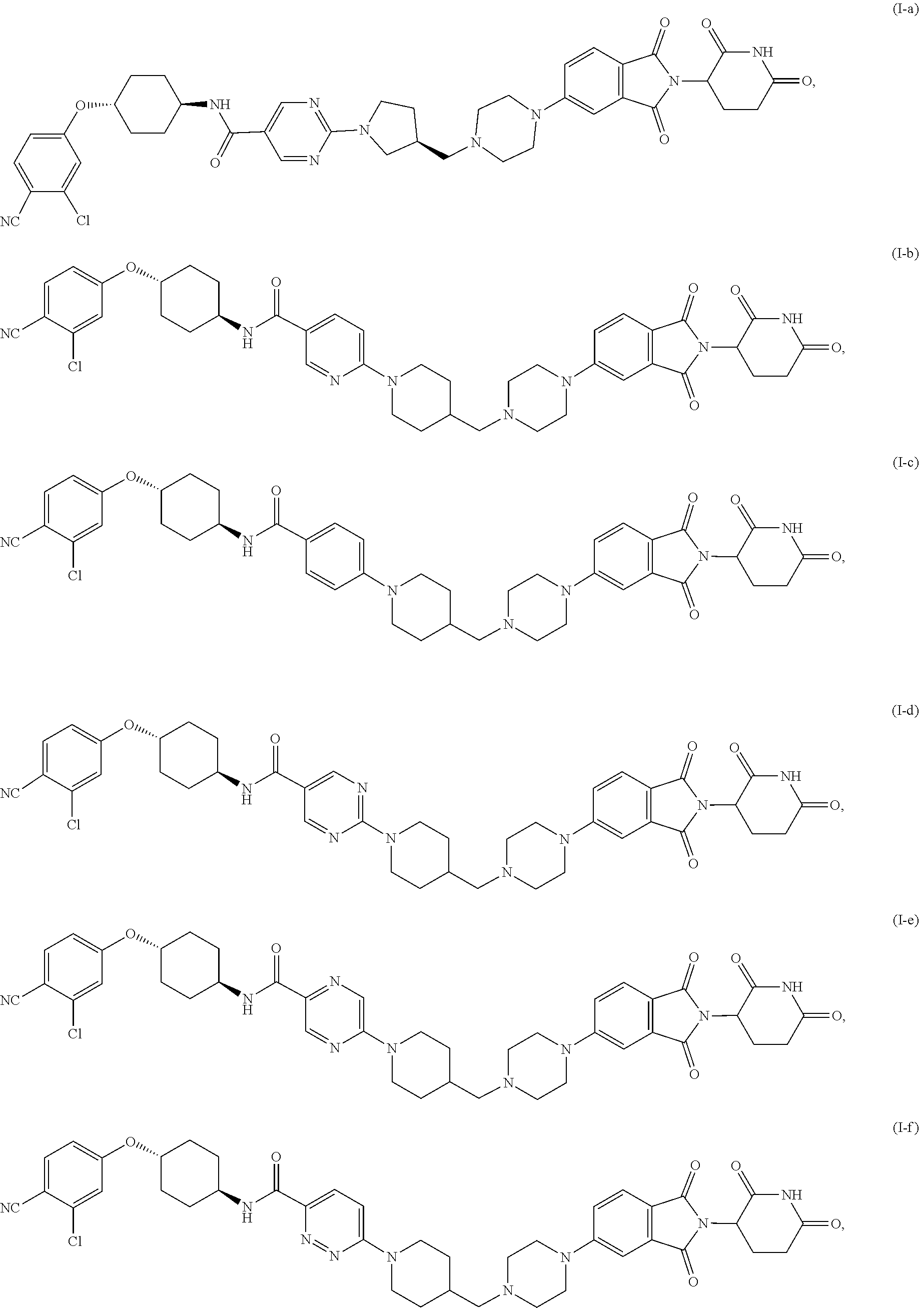

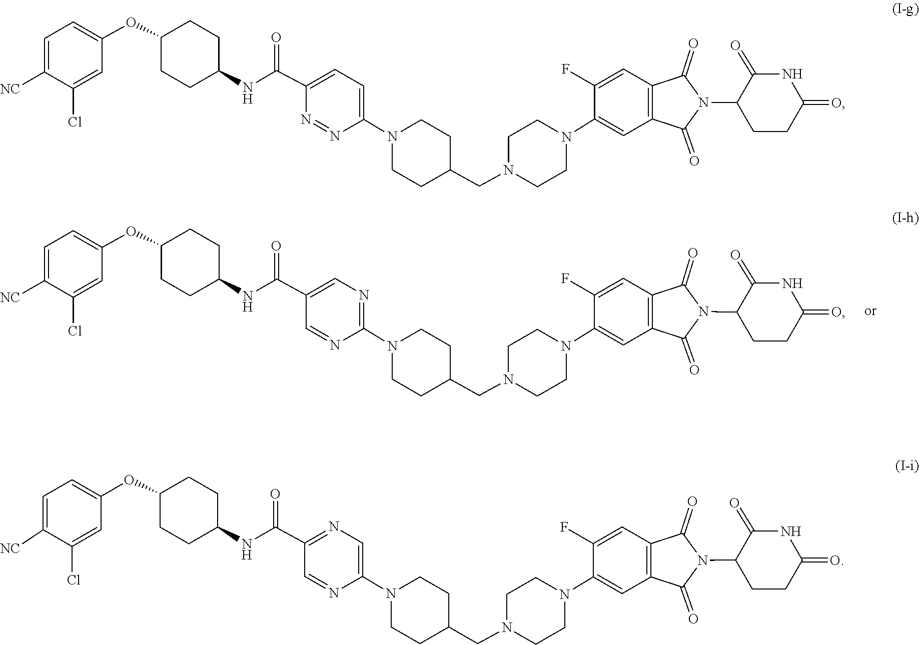 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "or" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

14. The method of claim 1 or 2, wherein the compound of Formula (I) is administered orally to the subject.

15. The method of claim 1 or 2, wherein the therapeutically effective amount of the compound of Formula (I) is administered to the subject once a day, twice a day, three times a day, or four times a day.

16. The method of claim 15, wherein the therapeutically effective amount of the compound of Formula (I) is administered to the subject once a day.

17. The method of claim 1 or 2, wherein the therapeutically effective amount of the compound of Formula (I) is administered to the subject all at once or is administered in two, three, or four portions.

18. The method of claim 1 or 2, wherein the therapeutically effective amount of the compound of Formula (I) is about 70 mg to about 1000 mg.

19. The method of claim 18, wherein the therapeutically effective amount of the compound of Formula (I) is about 100 mg to about 280 mg.

20. The method of claim 1 or 2, wherein the therapeutically effective amount of the compound of Formula (I) results in a mean day 15 AUC₀₋₂₄ of greater than about 4,500 ng*hr/mL, about 4,600 ng*hr/mL, about 4,700 ng*hr/mL, about 4,800 ng*hr/mL, about 4,900 ng*hr/mL, about 5,000 ng*hr/mL, about 5,100 ng*hr/mL, about 5,200 ng*hr/mL, about 5,300 ng*hr/mL, 5,400 ng*hr/mL, about 5,500 ng*hr/mL, about 5,600 ng*hr/mL, about 5,700 ng*hr/mL, about 5,800 ng*hr/mL, about 5,900 ng*hr/mL, or about 6,000 ng*hr/mL.

21. The method of claim 20, wherein the therapeutically effective amount of the compound of Formula (I) results in a mean day 15 AUC₀₋₂₄ of greater than about 4,500 ng*hr/mL and less than about 5,500 ng*hr/mL.

22. The method of claim 1 or 2, wherein the therapeutically effective amount of the compound of Formula (I) results in a mean day 15 Cₘₐₓ of greater than about 300 ng/mL and less than about 400 ng/mL.

23. The method of claim 18, wherein the therapeutically effective amount of the compound of Formula (I) results in a mean day 15 Cₘₐₓ of greater than about 330 ng/mL, about 335 ng/mL, about 340 ng/mL, about 345 ng/mL, about 350 ng/mL, about 355 ng/mL, about 360 ng/mL, about 365 ng/mL, about 370 ng/mL, about 375 ng/mL, or about 380 ng/mL.

24. The method of claim 1 or 2, wherein the compound of Formula (I) is formulated as a tablet.

25. The method of claim 24, wherein the tablet comprises a compound of Formula (I) and, optionally, one or more of the following: emulsifier; surfactant; binder; disintegrant; glidant; and lubricant.

26. The method of claim 1 or 2, wherein the subject is in a fed state.

27. The method of claim 1 or 2, wherein the subject is in a fasted state.

28. A method of treating prostate cancer in a subject in need thereof, comprising once a day, oral administration of a therapeutically effective amount of the compound of Formula (I), or a pharmaceutically acceptable salt, enantiomer, stereoisomer, solvate, polymorph, isotopic derivative, or prodrug thereof, wherein the compound of Formula (I) is selected from the group consisting of: 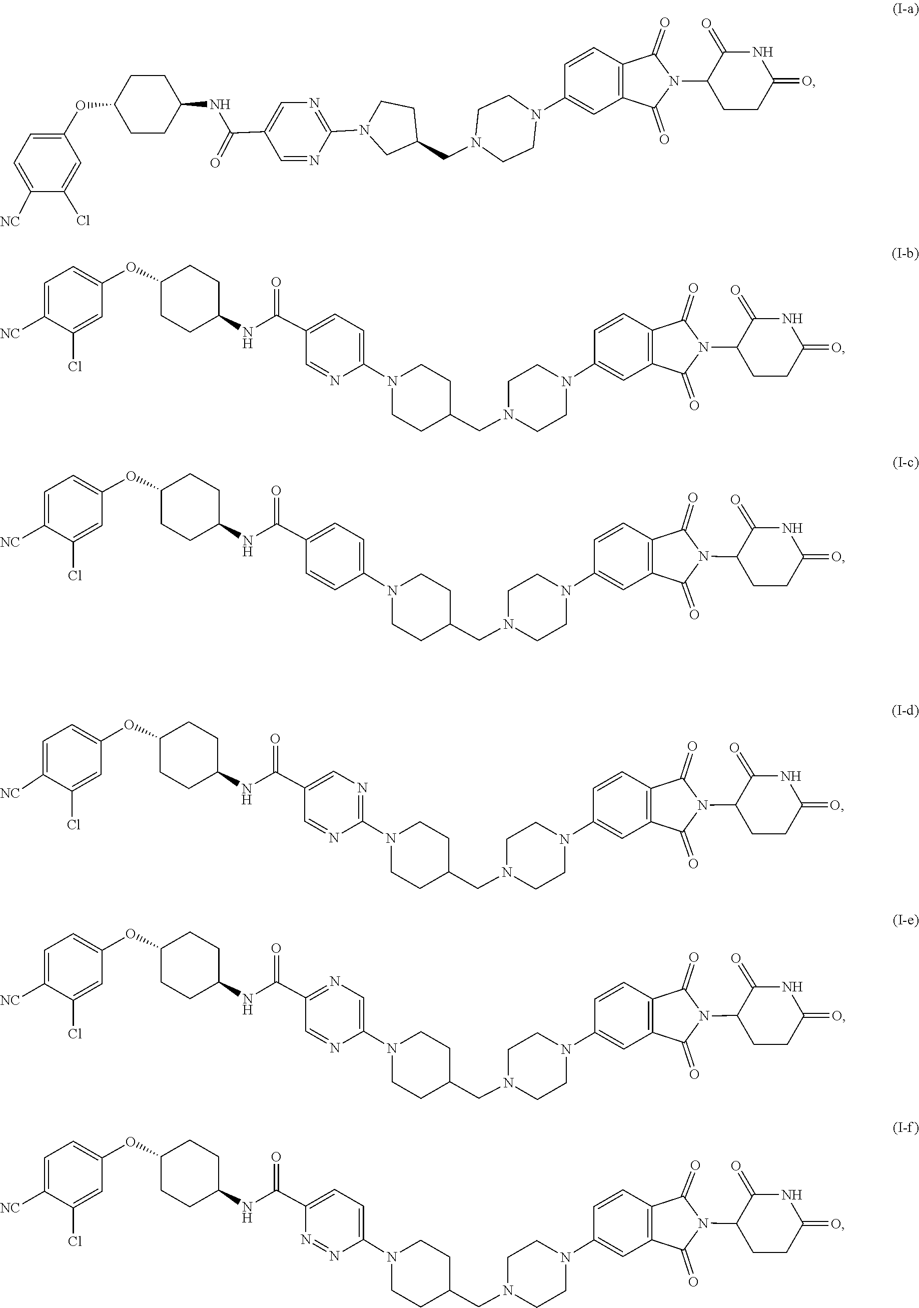 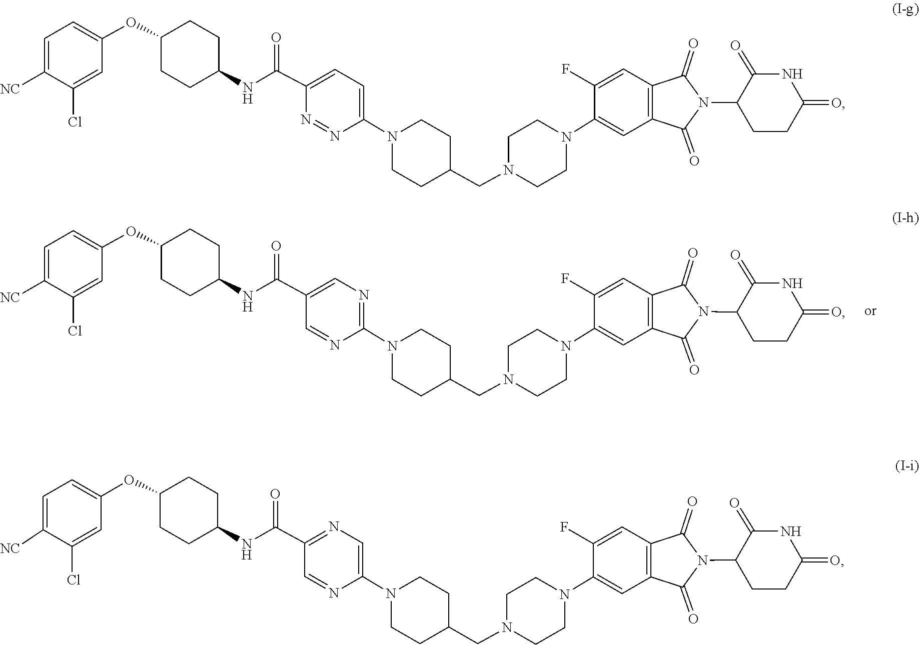 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "or" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]
or a pharmaceutically acceptable salt, enantiomer, stereoisomer, solvate, polymorph, isotopic derivative, or prodrug thereof, further comprising the step of discontinuing or reducing the administration of rosuvastatin to the subject prior to initiating administration of the compound of Formula (I).

29. A method of treating prostate cancer in a subject in need thereof, comprising once a day, oral administration of a therapeutically effective amount of the compound of Formula (I), wherein the compound of Formula (I) is selected from the group consisting of: 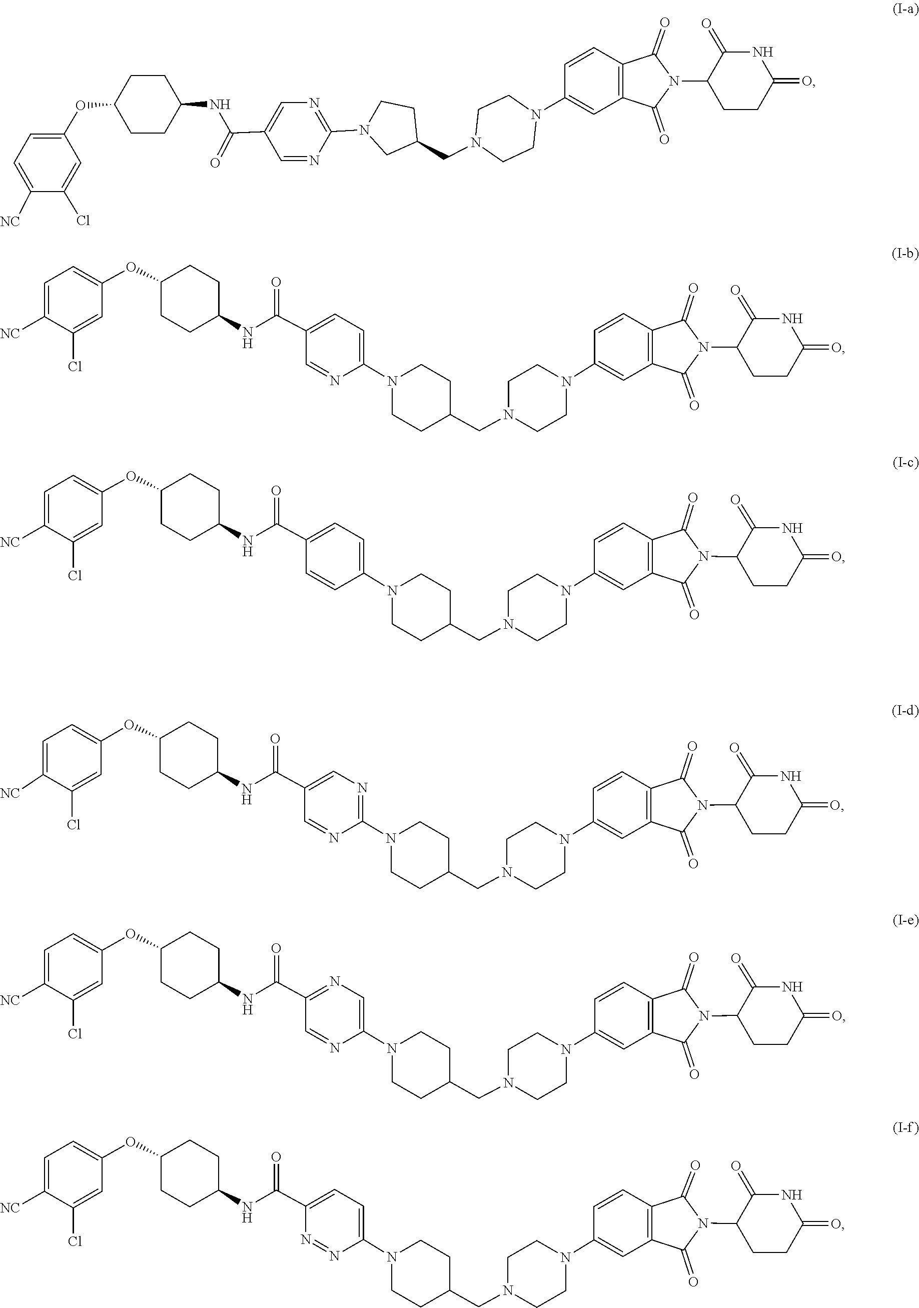 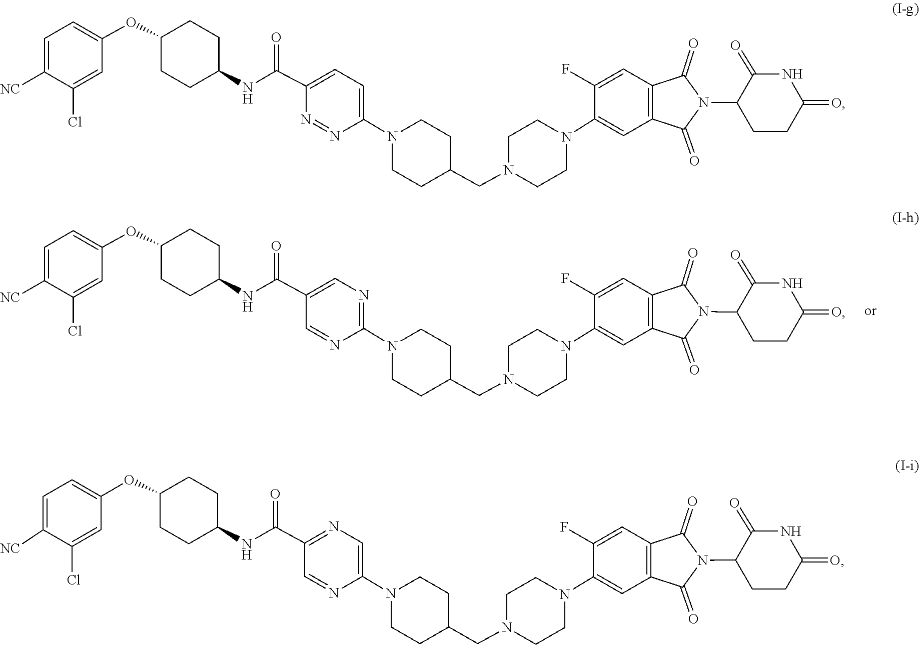 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "or" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]
further comprising the step of discontinuing or reducing the administration of rosuvastatin to the subject prior to initiating administration of the compound of Formula (I).

30. The method of claim 28 or 29, wherein the therapeutically effective amount of the compound of Formula (I) is administered to the subject all at once or is administered in two, three, or four portions.

31. The method of claim 28 or 29, wherein the therapeutically effective amount of the compound of Formula (I) is about 70 to about 1000 mg.

32. The method of claim 28 or 29, wherein the compound of Formula (I) is formulated as a tablet.

33. The method of claim 28 or 29, further comprising the step of discontinuing the administration of rosuvastatin to the subject prior to initiating administration of a therapeutically effective amount of the compound of Formula (I), or a pharmaceutically acceptable salt, enantiomer, stereoisomer, solvate, polymorph, isotopic derivative, or prodrug thereof.

34. The method of claim 28 or 29, further comprising the step of reducing the administration of rosuvastatin to the subject prior to initiating administration of a therapeutically effective amount of the compound of Formula (I), or a pharmaceutically acceptable salt, enantiomer, stereoisomer, solvate, polymorph, isotopic derivative, or prodrug thereof.

35. The method of claim 33, wherein the administration of rosuvastatin is discontinued in the subject beginning at a time point prior to initiating the administration of compound (I), or a pharmaceutically acceptable salt, enantiomer, stereoisomer, solvate, polymorph, isotopic derivative, or prodrug thereof, wherein the time point is at least the longer of 5 days or 5 half-lives of rosuvastatin.

36. The method of claim 34, wherein the administration of rosuvastatin is reduced in a subject taking rosuvastatin beginning at a time point prior to initiating the administration of compound (I), or a pharmaceutically acceptable salt, enantiomer, stereoisomer, solvate, polymorph, isotopic derivative, or prodrug thereof, wherein the time point is at least the longer of 5 days or 5 half-lives of rosuvastatin.

37. The method of any one of claim 1, 2, 28, or 29, wherein the compound of Formula (I) is the compound (I-g).

38-44. (canceled)
